(19) United States
(12) Patent Application Publication
(10) Pub. No.: US 2023/0117365 A1
(43) Pub. Date: Apr. 20, 2023

(54) Back Pillow and Back-Mounted Support Member for Measuring Body Parameters during Sleep and Facilitating Side Sleeping Orientation

(71) Applicant: Family Concepts II, LLC, Escondido, CA (US)

(72) Inventor: Vaughn W. North, Escondido, CA (US)

(21) Appl. No.: 17/794,594

(22) PCT Filed: Jan. 25, 2021
(86) PCT No.: PCT/US2021/014973

§ 371 Date: Jul. 21, 2022

Related U.S. Application Data
(60) Provisional application No. 62/965,131, filed on Jan. 23, 2020.

(51) Int. Cl. A47C 20/02; A61B 5/00; A61B 5/11; A61B 5/08

(52) U.S. Cl. CPC A47C 20/027; A61B 5/6831; A61B 5/1116; A61B 5/1117; A61B 5/0806; A61B 5/0816; A61B 5/6823; A61B 5/6819

(57) ABSTRACT

A back-mounted support device provides a stable platform for various monitoring and position support devices. The support device comprises a stiffening member configured for placement along a user's spine, the stiffening member extending from a top end to be positioned between the user's shoulder blades to a bottom end to be positioned along the spine and near a waistline of the user. A shoulder attachment system is coupled to the stiffening member to stabilize an upper portion of the stiffening member. A waist attachment system is coupled to the stiffening member to stabilize a lower portion of the stiffening member. The shoulder and waist attachment systems are configured to stabilize the stiffening member along the spine, while minimizing irritating body contact. Monitoring devices may be mounted anywhere on the support device for facilitating monitoring of body parameters.

### CROSS-REFERENCE TO RELATED APPLICATIONS

This application claims the benefit of U.S. Provisional Patent Application No. 62/965,131, filed Jan. 23, 2020, which is incorporated by reference herein in its entirety

### BACKGROUND OF THE INVENTION

Modern trends with telemedicine are demonstrating the value of frequent monitoring of health parameters of individuals. Particularly needful are seniors who are discharged from a hospital, but that require some degree of monitoring as a preventative measure to avoid having to be readmitted to the hospital at a high cost. Typically, measurements obtained from such monitoring are made over short time intervals during the day and with the patient alert and awake. Measuring and documenting blood pressure, heart rate and oxygen saturation of patients on routine bases can be valuable; however, such data taken over brief periods may offer an incomplete and/or ambiguous status of patient health.

Many patients are stable while awake, but deteriorate profoundly during sleep. During sleep, they may in fact develop life threatening conditions that are not suspected by clinicians or that are not manifest or apparent during short term measurements while awake. Sleep disordered breathing with hypoxemia or unstable breathing patterns, for example, is extremely common and frequently overlooked, even in patients who do not complain or who are not aware of snoring or other sleep problematic symptoms. Accordingly, one cannot depend on symptoms (or the absence of symptoms) exhibited When a patient is awake to make a clinical decision regarding physiologic status during sleep.

As an example of this issue, patients discharged from a hospital on opioids may not register indicators of risk while awake, as compared to the very unstable breathing patterns that can develop in some of these patients during sleep. As another example, patients with underlying cardiopulmonary disease (e.g. COPD), congestive heart failure, or those recovering from pneumonia may be stable and exhibit normal conditions while awake, but such patients can exhibit severe respiratory insufficiency while asleep. Despite these potentially life-threatening risks, current practices pertaining to patient monitoring and the obtaining of measurements during monitoring events focus on patients that are ambulatory and awake, and therefore do not address or account for these risks. Although it would be beneficial to supplement current patient monitoring and associated measurements with measurements taken while patients are asleep, taking such measurements (e.g., vital signs) poses additional control and stability problems. For example, data retrieval can be difficult from a patient that may frequently be moving about during sleep, as well as one that is often interrupted by restlessness and periods of wakefulness.

When considering the needs of the larger, general population, the ever growing complexity and stress of modern life suggests the need for increased attention on evaluating sleep as an essential component of ongoing medical care. An increasing number of devices are emerging in the marketplace for tracking sleep quality, as well as accessing biometric information associated with individual sleep patterns. In addition to obtaining data on blood pressure, oximetry, pulse rate, glucose levels, respiration patterns and other biometric parameters, it is well known that sleeping position and orientation can be an important factor in assessing sleep issues that need therapeutic attention. For example, it is now recognized that side sleeping can be an effective adjunct therapy to continuous positive airway pressure (CPAP) usage for apnea patients as well as for patients exhibiting other forms of sleep disturbance.

Although clinical and home-based sleep study programs are becoming more common, part of the challenge practitioners face involves minimizing the physical encumbrance of the numerous test devices and equipment on the body and the array of connecting hardware and wiring which the patient is required to be connected to so that the patient is able to experience as close to a normal (i.e., normal as it pertains to that particular patient) sleep event as possible. Indeed, it is not uncommon for a patient who undergoes a formal sleep study to toss and turn all night due to being entangled with wires, tubes, electrodes, chest and body belts, a nose cannula and finger devices (such as those for measuring oxygen saturation and flow rates). For many patients this experience is anything but restful and may seem of questionable value as a representation of their normal sleep pattern. Typical problems include the gradual displacement of this testing equipment across the body with each turn and twist, as well as wrestling with wires and tubes caught in a hand or arm. This is particularly true with home study systems that do not rely on adhesive patches and sensors that remain fixed on the patient's body. In addition, there is usually a system control case and manifold package that is suspended on a strap on the chest for receiving input from the various tubes, wires and sensors applied across the body. The manifold generally has significant weight that causes it to shift in location as the body turns and rolls in the bed. It is common for an accelerometer or position sensor to be housed in this package, thus undesirable displacement of the manifold can create difficulty in maintaining a fixed reference position on the body, thereby compromising a needed point of reference for base line orientation.

Accordingly, a need exists to develop methods and devices that can stabilize body sensors and other testing components in a secure and predictable arrangement on the body during sleep. The mere shifting of various test devices across the body during sleep can create patient anxiety regarding the accuracy of such sensor or control hardware. This is particularly true of sensors for measuring body position and orientation where part of the test requires a durable point of reference to assess vertical side sleeping versus supine position. Furthermore, maintenance of proper location of sensors on the body (such as a nose cannula for respiration) can be a matter of both comfort and effectiveness in data accuracy.

Sleep positional orientation is also an important factor for many persons in preserving health, particularly for individuals having adverse sleep conditions such as sleep apnea, snoring, insomnia, and various breathing irregularities. Monitoring the state of health and various body parameters and biometrics of an individual can be an important component of health care, particularly for seniors who have become more susceptible to age-related health conditions. There is now general acknowledgement by the medical community that many people, particularly older people, enjoy better sleep when on their side, avoiding supine sleeping orientation. This is particularly true for persons suffering from positional sleep apnea and snoring.

During times of sleep in a semi-consciousness or unconsciousness state, the position of the body is seldom within the person's active awareness. One may be changing positions among basic orientations of lying on one's back (supine), left side, right side, and front. In addition, there are transitional positions between each of these basic positions (inclined partially on the back and on a left or right side, etc.) that further define a continuum of position orientations for the human body while in a bed-rest setting.

### SUMMARY OF THE INVENTION

The present disclosure sets forth various stable platforms for use on the body that are capable of facilitating attachment of monitoring and other test devices in substantially fixed locations relative to the user, where the platforms are intended to be both highly functional as well as comfortable. This present disclosure sets forth a number of devices and techniques for accomplishing these objectives. Generally, these can be related to a back-mounted support structure worn on the body and supported in a manner that facilitates proper positioning and maintenance of test equipment in place, such as during sleep. The present disclosure sets forth a back-mounted support device to be worn on a user's back, shoulders and waist for providing a stable and comfortable mounting platform for various monitoring devices capable of determining a state of health and for registering body parameters, such as during sleep, including a body position or orientation sensor for recording sleeping positions and/or orientations for measuring side-sleep versus supine sleep. The position sensor(s) can be supported on the shoulder attachment member(s), the waist attachment member(s) or the stiffening member in a manner that takes advantage of the stability features of the back-mounted support device for maintaining a valid point of reference for orientation of the body as it moves. A rotatable elongate back pillow may also he advantageously coupled to the back-mounted support device where the back pillow can automatically move into opposing side-blocking positions on the upper body for preventing the supine sleep orientation, as well as for favoring other body positions for medical reasons during sleep.

In one example, the present disclosure sets forth a back-mounted support device for providing a stable platform for attachment of various monitoring and position control devices on a human body for enabling ongoing measurement of body parameters of an individual user located on a bed. in addition, it is adaptable for sustaining a desired body orientation on the bed, such as a side-sleeping position or other positions where body injury or sensitivity requires minimizing pressure on the affected area. A principle component is an elongate stiffening member configured for placement along a user's spine. The stiffening member has a length and limited width including (i) a top end to be positioned substantially between shoulder blades of the user and (ii) a bottom end to be positioned along the spine and near a waistline of the user. This limited width at the shoulder blades provides greater comfort to the user and assists with long term compliance in use. The stiffening member is configured through use of compositional stiffness and geometric shape for generally maintaining a stabilized position proximate at the user's back and along the spine.

A shoulder attachment member is configured for attachment at shoulders of the user and to the top end of the stiffening member to stabilize an upper portion of the stiffening member in a position along the user's upper spine. A waist attachment member is attached near the waist of the user and coupled to the bottom end of the stiffening member to stabilize a lower portion of the stiffening member in a position along the user's lower spine in a substantially centered location of the user's low back. The combination of shoulder and waist attachment members helps to stabilize the stiffening member substantially along the spine during sleep, while minimizing irritating body contact. At least one body position sensor is coupled in a substantially fixed positional relationship therewith and proximate to the user's back to monitor body movement as well as orientation with respect to various side-versus-supine positions of the body during sleep. Other monitoring components may be formed or attached as part of the back-mounted support structure, including at least one airflow respiration monitoring sensor (ARMS) with a control component mounted at the waist attachment member or the shoulder attachment member with at least one remote sensor component interconnected to the control component and the ARMS by a tubular interconnect line. This line may be mounted along the stiffening member and/or along the shoulder attachment member. The remote sensor may include a nose cannula or similar device for measuring breath pressure through the interconnect. Respiration may also be measured by a remote tension sensor component (RTSC) including a tension sensor coupled to at least one chest respiration band which is mounted at the stiffening member to provide a stable anchor point for the band with respect to the user and having an adjustable length which can be applied in tension around the user's chest and/or abdomen for monitoring respiration expansion.

An example of a body positioning component for the present invention includes a back-mounted. pillow device and method for enabling an individual lying on a bed to sustain a preferential side-sleeping orientation on either a left or right side. The device comprises an elongate, tubular shaped pillow having opposing top and bottom ends and. being attached to and supported by a light weight, elongate stiffening support member configured for suspending the pillow in placement along a user's spine. The top and bottom ends of the elongate pillow are respectively coupled at top and bottom ends of the stiffening member by first and second interconnecting attachment members, each having an. attachment length which provides for free rotation of the pillow between opposing rest positions along the user's back at right and left sides in response to gravity. This rotation is naturally synchronized. by gravity with movement of the body between alternating side-sleeping orientations.

### BRIEF DESCRIPTION OF THE FIGURES

Features and advantages of the invention will be apparent from the detailed description which follows, taken in conjunction with the accompanying drawings, which together illustrate, by way of example, features of the invention; and, wherein:

FIG. 1A illustrates an isometric view of a back-mounted support structure in accordance with an example of the present disclosure, the back-mounted support structure having an array of sensors, monitoring devices or components integrated into various parts of the back-mounted support structure, the back-mounted support structure further optionally including a position control device in the form of a back pillow, shown in phantom lines, supported by the stiffening member.

FIG. 1B illustrates a front, isometric view of the back-mounted. support structure of FIG. 1A, with the inclusion of the positioning back pillow.

FIG. 1C illustrates an isometric view of the stiffening member of the back-mounted support structure of FIGS. 1A-1B.

FIG. 2 illustrates an isometric view of a back-mounted support structure in accordance with an example, the back-mounted support structure being configured similarly as the back-mounted support structure of FIG. 1B, except without many of the monitoring devices and/or sensors supported thereon, wherein the back-mounted support structure functions primarily as a stable platform for the position control device, namely the back pillow.

FIG. 3 illustrates a rear, isometric view of the back-mounted support structure of FIG. 2.

FIG. 4 illustrates a side, isometric view of the back-mounted support structure of FIG. 2.

FIG. 5 illustrates a graphical end view representing the back-mounted support structure of either of FIGS. 1A-1B, 2-4 (this could also represent the back-mounted support structure of FIGS. 5A-5B) as attached to or donned by a user, with the user shown in a side-sleeping position

FIG. 6A illustrates a rear, isometric view of a back-mounted support structure in accordance with an example of the present disclosure, the back-mounted support structure having a stiffening member in the form of an elongate stiffening rod, and having an array of sensors, monitoring devices or components supported by various components of the back-mounted support structure for monitoring medical parameters associated with the user, the back-mounted support structure further including a position control device in the form of a back pillow.

FIG. 6B illustrates a front isometric view of the back-mounted support structure of FIG. 6B.

FIG. 7A illustrates an isometric view of an exemplary stiffening member in the form of an elongate rod operable with the back-mounted support structure of FIGS. 6A and 6B.

FIG. 7B illustrates an isometric view of an exemplary stiffening member in the form of an elongate rod operable with the back-mounted support structure of FIGS. 6A and 6B.

FIG. 8A illustrates the back-mounted support structure of FIGS. 6A and 6B as donned by a user, the stiffening member in the form of the elongate stiffening rod being curved and having an angled and arcuate configuration along its length for locating or displacing the elongate rod away from the user's back, the back-mounted support structure further comprising shoulder attachment members adjustably supported from the elongate stiffening rod.

FIG. 8B illustrates a detailed view of the adjustable mount for the shoulder attachment members of FIG. 8A.

FIG. 9 illustrates an isometric view of a decoy pillow for simulating blocking action provided by the back-mounted support structures of either of FIGS. 1A-1B, 2-4, 6A-6B or 8A-8B, in combination with knee and chest pillows.

FIG. 10 illustrates a graphical view of a patient monitoring system incorporating any of the back-mounted support structures of FIGS. 1A-1B, 2-4, 6A-6B or 8A-8B.

### DETAILED DESCRIPTION

As used herein, the term “substantially” refers to the complete or nearly complete extent or degree of an action, characteristic, property, state, structure, item, or result. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. In other words, something that is “substantially” free of an item or quality may still actually contain such item or quality as long as there is no measurable effect thereof. As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

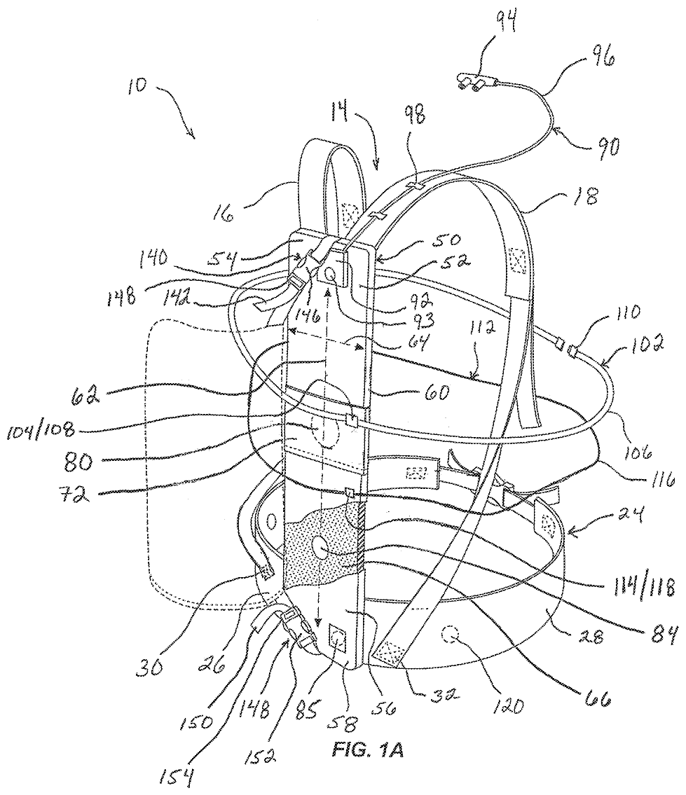

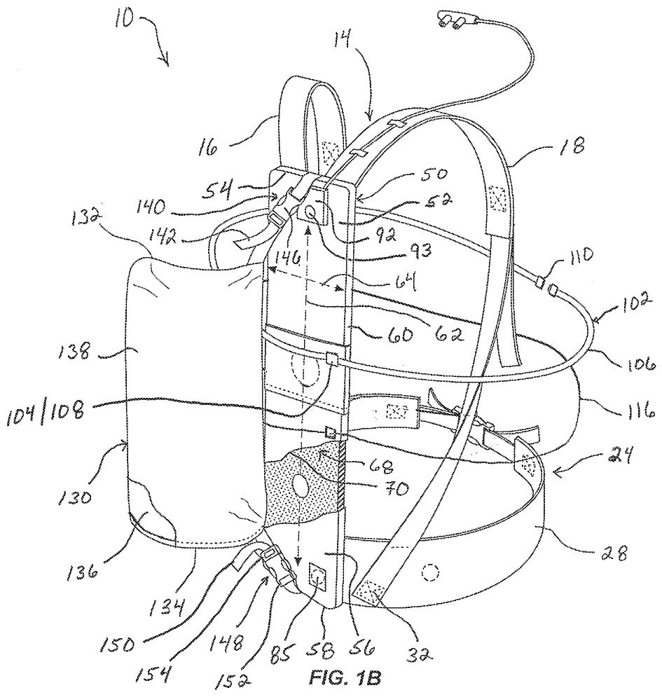

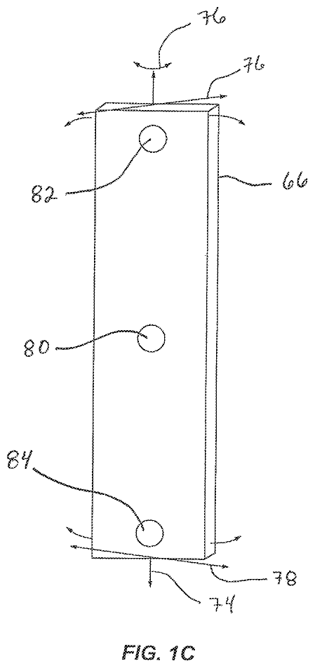

With reference to FIGS. 1A-1C, illustrated is a back-mounted support device 10 for providing a stable platform for attachment, positioning and/or support of various monitoring devices (e.g., see monitoring devices throughout) and one or more position control devices (e.g., see position control device in the form of a back pillow 130) to, on, and/or relative to a human body for a variety of health and health monitoring related purposes, in accordance with an example of the present disclosure. As will be discussed in more detail below, and as is applicable to all back-mounted support devices disclosed herein, the back-mounted support device 10 can be wearable by a user to facilitate the sensors, monitoring devices and/or the position control devices being located in respective positions, and stabilized in such positions, relative to the user. By being supported on the stable platform provided by the back-mounted support device 10, the sensors and monitoring devices are able to operate and perform as intended to generate accurate, reliable measurements associated with measuring and/or monitoring an aspect of or related to the user. Likewise, by being supported on the stable platform provided by the back-mounted support device 10, the position control device(s) operate and perform as intended to correctly and reliably control certain movements and positions or orientations of the user.

In one intended application, which is not intended to be limiting in any way, upon being donned by a user, the back-mounted support device 10 can provide a stable platform for a variety of monitoring devices, sensors, and/or a combination of these used to facilitate periodic, temporary or ongoing measurement of body parameters, movements, etc. of an individual user lying on a bed, such as during sleep. In another aspect of this particular application, upon also removably attaching and appropriately positioning a position control device, such as a back-pillow, the back-mounted support device 10 can further provide a stable platform for the position control device (the back pillow) operable to establish and sustain a desired body position and orientation of the user on the bed, such as a side-sleeping position. While other applications are contemplated, such as daytime monitoring with the user undergoing various activities, the back-mounted support device 10 (and the other back-mounted support device examples discussed herein) will primarily be discussed in the context of being used within the specific application of the user donning the back-mounted support device 10 to facilitate the monitoring of body and other parameters while the user is sleeping. As will be applicable in any application, with one or more monitoring devices supported, and/or with a position control device attached and supported, the back-mounted support device 10 (and the other back-mounted. support device examples discussed herein) can provide a stable platform for such monitoring and position control devices so that their location on or relative to the body of the user remains substantially consistent and predictable. This facilitates uniform data recording conditions, such as across a night of movement on a bed during sleeping, thus leading to more accurate and reliable measurements that can assist practitioners in a variety of ways to care for, and in some cases improve the health of, the user.

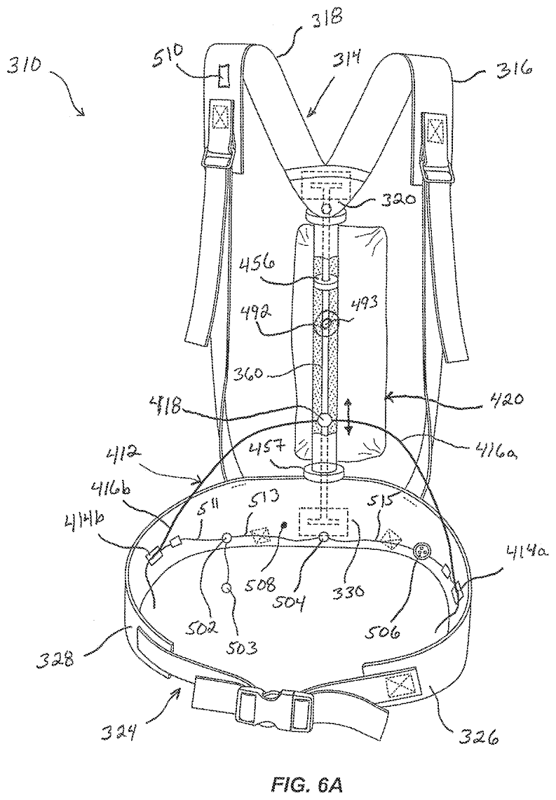

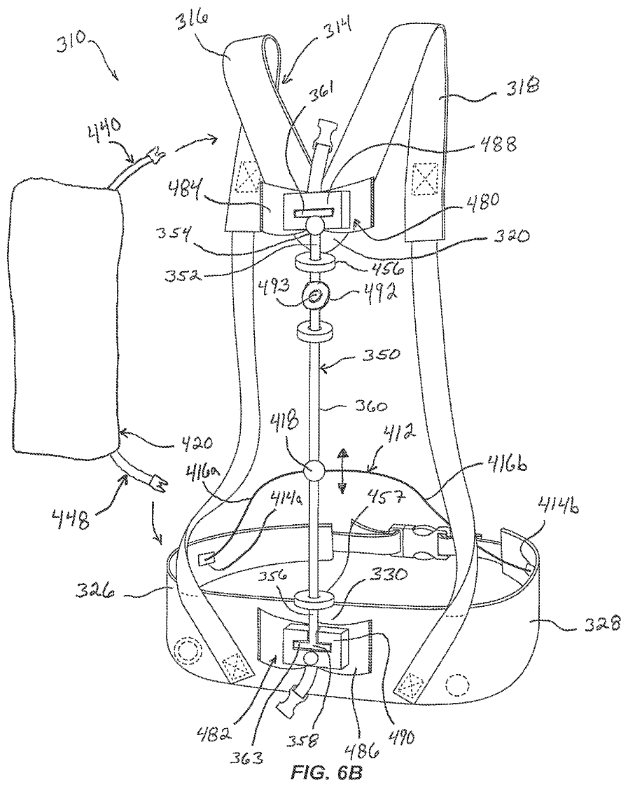

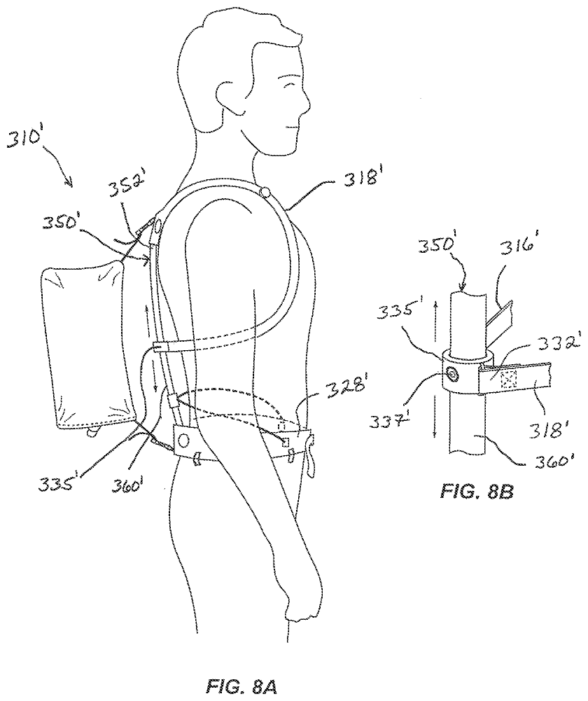

The back-mounted support device 10 can comprise a wearable harness-type of device having an arrangement of components configured to interface with and to facilitate attachment (i.e., wearing, donning, etc.) of the back-mounted support device 10 to an upper body of a human user (e.g., see FIG. 8, with the user donning the back-mounted support device 310′ of FIGS. 6A-6B). The back-mounted support device 10 can comprise a variety of different configurations and designs, some examples of which are discussed herein, but which are not intended to be limiting in any way. In one specific example, as shown, the back-mounted support device 10 can comprise wearable harness-type of device comprising a shoulder attachment system 14 operable to support the back-mounted support device 10 about or on the shoulders of a user, a waist attachment system 24 operable to support the back-mounted support device 10 about or on the waist of the user, and a stiffening member 50 operable to support and connect these together.

In one aspect, as shown, the shoulder attachment system 14 can comprise first and second shoulder attachment members 16 and 18 (e.g., in the form of shoulder straps or shoulder harnesses). However, the shoulder attachment system 14 can comprise other configurations, such as a single shoulder attachment member having first and second shoulder straps. In any event, reference to shoulder attachment members 16, 18 is intended to encompass each of these configurations. Likewise, the waist attachment system 24 can comprise first and second waist attachment members 26 and 28 (e.g., in the form of waist straps or waist harnesses). However, the waist attachment system 24 can comprise other configurations, such as a single waist attachment member having first and second waist strap members integrally formed that connect together at respective ends, as shown (e.g., a belt-like structure having connectable end members). In any event, reference to waist attachment members 26, 28 is intended to encompass each of these configurations.

The shoulder attachment members 16, 18 can be configured for attachment to the user at the shoulders and which can extend partially along the upper back and chest of the user. More specifically, in one example, the shoulder attachment members 16, 18 can be configured to extend from a coupling or attachment location on an upper portion of the stiffening member 50, extend over the shoulders of the user, and wrap around the chest and under the arms of the user, where they are coupled to the waist attachment members 26, 28 or to the stiffening member 50. The shoulder attachment members 16, 18 can facilitate the carrying of a load from the back-mounted support device 10, and can interface with the user's body to help facilitate a stable platform relative to the user's body as provided by the back-mounted support device 10, as discussed herein. The shoulder attachment members 16, 18 can comprise a variety of sizes and configurations, and these can vary depending on the shape and size of the user's body.

The waist attachment members (e.g., joinable waist straps) 26, 28 can be configured for attachment to the user at or near the waist of the user. The waist attachment members 26, 28 can also facilitate the carrying of a load from the back-mounted support device 10, and can interface with the user's body to help generate, along with the shoulder attachment members 16, 18, a stable platform relative to the user's body as provided by the back-mounted support device 10. The waist attachment members 26, 28 provide a belt-like support around the user's waist coupled to the bottom end 58 of the stiffening member 50, wherein the waist attachment members 26, 28 are configured to stabilize a lower portion of the stiffening member 50 in a position along the user's spine in a centered location of the user's lower back. This helps minimize rotation and twisting of the stiffening member 50 and displacement away from the centered location at the spine. The waist attachment members 26, 28 can comprise a variety of sizes and configurations, and these can vary depending on the shape and size of the user's body.

in some aspects, the shoulder attachment members 16 and 18 and the waist attachment members 26, 28 can comprise respective adjustment members (e.g., buckles, etc.) that enable an individual length of these to be adjustable around the trunk of the user's body. Indeed, one or more of the shoulder attachment members 16, 18 and/or the waist attachment members 26, 28 may need to be adjusted for the purpose of establishing and maintaining proper, stable positioning of any sensors, monitoring devices and/or position control devices supported on the back-mounted support device 10, as well as to meet any comfort needs of the user.

The back-mounted support device 10 can further comprise an elongate stiffening member 50 configured for placement proximate the back of a user and along the user's spine. The purpose and function of the stiffening member 50 will be apparent from the description below.

The stiffening member 50 can be suitably sized and configured to minimize contact on the user's back in order to provide the needed comfort while minimizing irritation to the user, as well as to be stably positioned substantially between the shoulder blades and along the spine of the user so that it doesn't inadvertently displace out of position (e.g., wander significantly across the back) with body movement during sleep. The particular size, shape and material makeup of the stiffening member 50 can vary depending upon a number of different factors, such as the size of the user, a selected level of comfort desired by the user, performance effectiveness, and other factors. In one example, the stiffening member 50 can comprise a flat, rectangular configuration comprising an upper portion 52 defining a top end 54, a lower portion 56 defining a bottom end 58 and a bridging or spanning portion 60 extending between the top and bottom ends 54, 58, the stiffening member defining, at least in part, a length 62 and a width 64.

The stiffening member 50 can be coupled to, and between, the shoulder attachment members 16, 18 and the waist attachment members 26, 28, such that the stiffening member 50 extends between these. As shown, upper or proximal ends of the shoulder attachment members 16, 18 can be coupled to the stiffening member 50 at a location proximate the upper portion 52 (e.g., at the top end 54) of the stiffening member 50 to stabilize the upper portion of the stiffening member 50 in a position relative to the user's back, and along the user's upper spine. The waist attachment members 26, 28 can be coupled to the stiffening member 50 proximate the lower portion 56 (e.g., at the bottom end 58) of the stiffening member 50 to stabilize the lower portion of the stiffening member 50 in a substantially centered position of the user's lower back along the user's lower spine. As such, in one example, the length 62 can be sized and configured such that the top end 54 is positioned along the spine and substantially between the shoulder blades of the user, and such that the bottom end 58 is positioned along the spine and near a waistline of the user, wherein the stiffening member 50 extends between the top end 54 and the bottom end 58 from the shoulder blades of the user (from the upper spine portion of the user) to the waistline of the user (to the lower spine portion of the user), However, this particular size and length and configuration is not intended to be limiting in any way. Indeed, the stiffening member 50 can comprise other lengths as will be recognized by those skilled in the art depending upon, for instance, the intended use of the back-mounted support device 10, the physiology of the user, and other factors. The width 64 of the stiffening member 50 can be such that the stiffening member 50 is positionable between the shoulder blades of the user. With the stiffening member 50 maintained or stabilized in this position during use, the user can experience enhanced comfort and maintenance of long-term compliance in use as compared to a stiffening member 50 sized to extend beyond the shoulder blades. In one aspect, the stiffening member 50 can comprise a constant width 64 ranging from 1 to 6 inches. In another aspect, the width 64 of the stiffening member 50 can range between 2 to 4 inches. In another aspect, the stiffening member 50 can comprise a variable width 64 along its length. For example, the top and bottom ends 54, 58 can have a wider configuration than the spanning portion 35 extending between the top and bottom ends 54, 58. In another aspect, the stiffening member 50 can comprise a plurality of segments. Those skilled in the art will recognize that the stiffening member 50 can comprise a variety of sizes and configurations, and thus those described herein and shown in the drawings are not intended to be limiting in any way.

Lower or distal ends of the shoulder attachment members 16, 18 can be attached to the waist attachment members 26, 28. In another aspect, the distal ends 30 and 32 of the shoulder attachment members 16, 18, respectively, can be attached to the stiffening member 50. In either case, the shoulder attachment members 16, 18 can be secured, such that counter resistive forces are generated in the back-mounted support device 10 that act relative to one another so that both the shoulder attachment members 16, 18 and the waist attachment members 26, 28 can be put into tension, and so that the stiffening member 50 can be stabilized about the user's back along the spine (either in a position against the user's back, or offset from the user's back depending upon a configuration of the back-mounted support device 10).

The combination of the shoulder attachment straps 32, 31 and the waist attachment members 26, 28 function to enable the back-mounted support device 10 to be properly fit (e.g., to provide a snug fit) against and to the user's body. In addition, the shoulder attachment members 16, 18 and the waist attachment members 26, 28 are configured to operate together to help stabilize a positon of the stiffening member 50 substantially along the spine of the user, such that forces acting on the stiffening member 50 that may otherwise cause subsequent displacement of the stiffening member 50 relative to the user's back to occur are resisted. Such forces acting on the stiffening member 50 can be induced by certain movements of the user, such as when moving during sleep. Stabilizing the stiffening member 50 relative to the user's back also functions to minimize irritating or intrusive and unpleasant contact with the user's body. In practice, the user can don the back-mounted support device 10 by inserting his/her arms through the shoulder attachment members 16, 18, and then fastening the waist attachment members 26, 28 together around the waist. Either prior to or after donning the back-mounted support device 10, each of the shoulder attachment members 16, 18 and the waist attachment members 26, 28 can be individually adjusted, via their respective one or more adjustment members, to position the stiffening member 50 in a proper and functional stable position, wherein the shoulder attachment members 16, 18, the waist attachment members 26, 28 and the stiffening member 50 enable the back-mounted support device 10 to provide a stable support platform for the sensors, monitoring devices and position control devices supported thereon.

The respective distal ends of the shoulder attachment members 16, 18 can be attached at any attachment location along the length of the waist attachment members 26, 28 or the stiffening member 50. However, attaching the distal ends 30, 32 of the shoulder attachment members 16, 18 at an attachment location that, with the back-mounted support device 10 donned by a user, is rearward of a mid-coronal plane that transects the body of the user into two halves, namely a front half and a back half (i.e., anterior and posterior halves, or ventral and dorsal halves) can operate to draw the shoulder attachment members 16, 18 and the top portion or end 14 of the stiffening member 50 inward toward one another, at least to some degree, upon the shoulder attachment members 16, 18 being adjusted to the appropriate tension (e.g., tightened around the user's body) to achieve the stable platform function. Such an attachment location can enhance the stability of the stiffening member 50 due to the direction of forces acting on the stiffening member and against the body of the user by the shoulder attachment members 16, 18 and the waist attachment members 26, 28 compared with the direction of forces resulting from an attachment location forward the mid-coronal plane. Moreover, with an attachment location rearward of the mid-coronal plane, longitudinal forces acting along a longitudinal axis of the portion of the shoulder attachment members 16, 18 beneath the user's arms and passing through the mid-coronal plane along respective lateral sides of the user (which forces are induced by the attachment of the shoulder attachment members 16, 18 to the waist attachment members 26, 28 or the stiffening member 50 and the user donning the back-mounted support device 10 (as appropriately adjusted), can be caused to be applied in a direction transverse to the mid-coronal plane. It is noted that the relative distance that the shoulder attachment portions 32, 34 and the upper portion 52 and top end 54 of the stiffening member 50 will be drawn inward toward one another as a function of the distance that the stiffening member 50 has a tendency to displace in a vertical direction upon the shoulder attachment members 16, 18 being adjusted and tightened against the user's body, will depend upon the vertical attachment location of the distal ends 30, 32 of the shoulder attachment members 16, 18 to the waist attachment members 26, 28 or the stiffening member 50 along a vertical axis (normal to ground if the user were standing), and a measured distance between the attachment location for the distal ends of the shoulder attachment members 16, 18 and the attachment location of the proximal ends of the shoulder attachment members 16, 18 at the upper portion 54 of the stiffening member 50. Furthermore, when the waist attachment members 26, 28 are suitably adjusted and their respective ends are secured to one another around the waist of the user, the resulting tension force generated within the waist attachment members 26, 28 operates to draw the bottom portion 56 and bottom end 58 of the stiffening member 50 inward towards the user's back. With the shoulder attachment members 16, 18 and the waist attachment members 26, 28 so configured. relative to the stiffening member 50 and the body of the user, the stiffening member 50 can be caused to be disposed in a more stable position relative to the user's back along the spine, while providing a greater resistance to subsequent displacement of the stiffening member 50 as a result of the more directionally inward forces induced within the back-mounted support device 10 made possible by the attachment location for the distal end of the shoulder attachment members 16, 18 being rearward of the mid-coronal plane as compared to an attachment location forward the mid-coronal plane (e.g., on a front portion of the waist attachment members 26, 28.

The stiffening member 50 can be formed of a variety of materials. However, the stiffening properties of the stiffening member 50 can be realized through use of either or both compositional stiffness (modulus of elasticity) and geometric size/shape, which stiffness properties help to generally maintain a stabilized position of the stiffening member 50 proximate the user's back and along the spine.

With respect to material makeup or composition of the stiffening member 50, in one example, as shown, the stiffening member 50 can be composed of or comprise a foam material. In one aspect, the foam can be configured as an insert 66 to be received and supported within a stiffening member compartment 68 of a sleeve 70 as part of the back-mounted support device 10, with the sleeve 70 and the foam insert 66 making up, at least in part, the stiffening member 50 of the back-mounted support device 10. The sleeve 70 can further comprise one or more compartments, pouches, pockets, etc. formed therein for receiving and supporting one or more monitoring devices on the stiffening member 50. For example, the sleeve 70 can comprise a pocket or pouch 72 sized and configured to receive a body positon sensor or other type of monitoring device (e.g., see body position sensor [] in dotted lines supported therein). With respect to the foam insert 66, the foam material can be configured to provide the stiffening member 50 with a suitable balance of stiffness versus flexibility. Indeed, the makeup of the foam material can be such that the stiffening member 50 is configured with a stiffness sufficient to resist twisting away from the user's back during use, thereby ensuring that the back-mounted support device 10 provides a stable platform for the monitoring and the position control devices, while also being sufficiently flexible so as to conform to the user's back along the spine so as to nest at the user's back with the top end 54 of the stiffening member 50 nested between or approximately between the shoulder blades. One example measure of suitable stiffness can be that the material of the foam insert 66 maintains a vertical orientation under its own weight, while possessing sufficient flexibility to enable the stiffening member 50 to conform to the surface of the user's back/spine, and while also possessing sufficient flexibility so as to enable different portions of the stiffening member 50 to be able to rotate (twist) in opposing directions along its longitudinal axis 74 (the vertical axis shown in FIG. 1C). This twisting rotation is depicted by arrows 76 representing rotation of the upper portion of the stiffening member 50 in one direction, and by arrows 78 representing rotation of the lower portion of the stiffening member 50 in an opposing direction, each about longitudinal axis 74. As discussed in more detail herein, the stiffening member 50 can thus facilitate the back-mounted support device 10 providing a stable platform for a plurality of body position or other monitoring devices supported about the stiffening member 50 or another part of the back-mounted support device 10 while also facilitating twisting out of plane upper and lower portions of the stiffening member 50 relative to one another. Thus, the stiffening member 50 operates to maintain its stability even during twisting movements by the user. Being able to provide a stable platform and also twist along a longitudinal axis provides significant benefits. For example, the shoulders of the user can be caused to rotate in one direction relative to the hips/waist of the user rotating in an opposing direction. This type of movement can occur in different situations, such as during sleep when the user's shoulders are rotated out of plane relative to his/her hips (e.g., the shoulders are rotated to some angular degree with the hips held in place, or the shoulders are rotated to a greater angular degree than an angular rotation of the hips). Such relative movement enables an upper body position sensor supported on an upper portion of the stiffening element 12 to measure and track an orientation of the shoulders relative to an orientation of the hips as measured by a lower body position sensor supported on a lower portion of the stiffening member 50. As such, the back-mounted support device 10 is capable of measuring the relative positions of two or more parts of the body of the user and maintaining a positional record of these. This can be beneficial in many patient monitoring situations, including, but not limited to, the monitoring of a user during sleep, where the back-mounted support device 10 enables evaluation of relative body positions of the user during sleep with respect to the occurrence of snoring and/or apnea events, and to identify any correlations between these.

The foam material can comprise any suitable type of stiff, closed cell or other foam or foam-like material. Other satisfactory stiffening component compositions can be selected as will be apparent to those having skill in the art, These can be selected and applied to realize the desired balance between flexibility and stiffness intended to be provided by the stiffening member 50. Examples include, but are not limited to, a thin plastic sheet or mesh material, a stiff fabric material, a thin aluminum member having a modest degree of flexibility and memory. A commercial product HL 384797 supplied by Hobby Lobby under the name “Silly Winks” satisfies these requirements.

The back-mounted support device 10 can further comprise one or more sensors or monitoring devices. As referred to herein, the term “monitoring device” refers to a sensor and/or a device incorporating or embodying or supporting or that is otherwise operable with one or more sensors that function to monitor one or more aspects of, or that are related to, the user. As shown, the back-mounted support device 10 comprises several different types of exemplary monitoring devices, each of which are operable from the stable platform provided by the hack-mounted support device 10. Each of the monitoring devices of the back-mounted support device 10 shown are described below. However, these are not intended to be limiting in any way as other types of monitoring devices in existence now or that may exist in the future are contemplated as being supportable by the back-mounted support device 10, as will be apparent to those skilled in the art.

As part of the function of the back-mounted support device 10 to provide a stable platform for structurally positioning various monitoring devices relative to the body of the user, the back-mounted support device 10 can further comprise one or more monitoring devices in the form of body position sensors (e.g., see body position sensor 80). The body position sensors can be coupled to or otherwise supported by the stiffening member 50, or other components of the back-mounted support device 10 in a substantially fixed positional relationship therewith and proximate to the user's back. The one or more body position sensors can be configured and can function to measure position and/or orientation of the user's body, and to provide body orientation data. For example, in one application, the body position sensor 80 can be used to detect a side sleeping orientation or a supine sleeping orientation of the user when sleeping.

The back-mounted support device 10 can comprise any number of body position sensors, and these can be located or positioned at any position on the back-mounted support device 10. In one example, the body position sensor(s) can be supported on the stiffening member 50 so as to locate the body position sensors proximate the user's back. As shown, the body position sensor 80 can be supported on or about the stiffening member 50, and can be positioned near a midpoint of the spanning portion 35 of the stiffening member 50. The body position sensor 80 can be coupled directly to the stiffening member 50 (e.g., via an adhesive, a hook and loop fastener, a clip, embedded in the material of the stiffening member 50, and others), or it can be otherwise supported in a fixed position on or about the stiffening member 50 (e.g., contained in a pocket or a sleeve of the stiffening member 50, in a pouch sewn to the stiffening member 50, and others). Additional body position sensors can be supported on the stiffening member 50, such as position sensors 82 and 84 (see FIG. 1C) positioned at the upper and lower portions 52, 56 (ear the top and bottom ends 54, 58) of the stiffening member 50, respectively. Supporting the body position sensors 80, 82 and 84 on the stiffening member 50 provides the ability to position these proximate the user's back and along the spine of the user. In addition, the stiffening member 50 provides an ideal location or base for the one or more body position sensors 80, 82 and 84 due the rotational and translational stability of the stiffening member 50 with respect to the body and trunk of the user. Location of one or more of the body position sensors 80, 82 and 84 can generally be at any stable position along the length of the stiffening member 50. Once one or more of the body position sensors 80, 82 and 84 are in place, these can be calibrated to register an initial or baseline reference position or orientation (e.g., a reference position or orientation with the user in a side sleeping orientation, which reference position or orientation should be substantially stable during sleep unless disturbed by movement). The body position sensors 80, 82 and 84 function to register changes in body orientation of the user (e.g., via the detection of a one position of a sensor relative to another position of the sensor). In the example application where the user is lying down, such as to sleep, registered changes in body orientation can be with respect to the reference frames of a mid-coronal plane corresponding to a parallel relationship with the user's back, and a sagittal plane corresponding to a perpendicular relationship with the user's back. Each of the body position sensors 80, 82 and 84 can be calibrated to register an initial or baseline reference point with the user in the desired side sleeping orientation. In the side sleeping orientation, the mid-coronal plane will be perpendicular or substantially perpendicular to ground (extending along a vertical axis), and the sagittal plane will be horizontal or substantially horizontal to ground (extending along a horizontal axis). However, in the event the user rolls away from a side sleeping orientation, such as to a supine orientation, the mid-coronal plane becomes horizontal or substantially horizontal with the ground, and the sagittal plane becomes perpendicular or substantially perpendicular relative to ground. In this situation, the body position sensors 80, 82 and 84 will register some change in body position. The registered change in each of the different body position sensors 80, 82 and 84 can be the same or it can be different, depending upon the degree of change in movement each undergoes.

One or more body position sensors can be supported on the shoulder attachment members 16, 18, or on the waist attachment members 26, 28, which in one aspect can provide an alternative sensor location to that of the stiffening member 50. In another aspect, additional body position sensors can be supported on the shoulder attachment members 16, 18 and/or the waist attachment members 26, 28, wherein the back-mounted support device 10 provides a full complement of body position sensors located at a number of different locations on the back-mounted support device 10.

Employing a plurality of body position sensors provides advantages over utilizing a single body position sensor, including, but not limited to, being able to provide a more complete and accurate movement history, being able to provide comparative data, such as data corresponding to relative shoulder and hip positioning and/or orientation (e.g., during sleep), and others. Such comparisons may be particularly useful when analyzing apnea events and relating such events to patterns of body position/orientation at various inclinations of side sleeping along the length of the spine. Indeed, multiple sensors supported at multiple different locations (such as at the upper, mid and lower portions of the stiffening member 50) can enable or facilitate concurrent position/orientation detection (and monitoring) for three-dimensional analysis, such as counter-rotational movement of the hips and shoulders of the user during sleep.

The body position sensor(s) can comprise any type of position and/or orientation sensor or device apparent to those skilled in the art, which can be supported on the back-mounted support device 10, and which be configured to measure or detect body position and/or orientation, such as with respect to various side versus supine positions of the body of the user during sleep.

As another example of the back-mounted support device 10 providing a stable platform for structurally supporting and positioning various monitoring devices on the stiffening member 50, the shoulder attachment members 16 and 18, and/or the waist attachment members 26, 28 and relative to the body of the user, the back-mounted support device 10 can further comprise a monitoring device in the form of one or more airflow respiration monitoring sensors (ARMS) (e.g., see airflow respiration monitoring device 90), wherein the ARMS device is capable of monitoring breath activated airflow rates of the user. The airflow respiration monitoring device 90 can comprise a control and sensing component 92 (i.e., a respiration monitor controller, having at least one sensor 93, a power supply, circuitry, processors and memory, transceiver, etc., these being operable to facilitate active or passive control of the ARMS device) mounted on one of the shoulder attachment members 16, 18, the stiffening member 50, and/or the waist attachment members 26, 28 (e.g., via a hook and loop fastener, an adhesive, or within a pocket formed in one of these). The airflow respiration monitoring device 90 can further comprise at least one remote receiving component 94, such as a conventional nasal cannula operable to deliver air flow to a sensor operable with or within the control and sensing component 92, by a tubular interconnect line 96 through which oxygen or air can flow. The tubular interconnect line 96 can comprise a flexible fluid delivery line similar to those used to convey oxygen or air to a patient from an oxygen or air source. At one end, the tubular interconnect line 96 can interface and connect with (e.g., fluidly connect) and extend away from the control and sensing component 92. The other end of the tubular interconnect line 96 can interface and connect with the remote receiving component 94. In order to control and manage the tubular interconnect line 96 so that it is out of the way of the user as much as possible, the tubular interconnect line 96 can be routed along a surface of the back-mounted support device 10 to a shoulder point or position on one of the shoulder attachment members 16, 18, where it can extend from the shoulder attachment member a sufficient distance to the user's nose. As shown, the tubular interconnect line 96 can be anchored at one or more anchor points on the back-mounted support device 10 as provided by one or more anchoring members. In one example, the anchoring members can comprise one or more looped members, such as looped members 98 attached to the shoulder attachment 32. The looped members 98 can be configured to receive and secure the tubular interconnect line 96 to the surface of the back-mounted support device 10. With this arrangement, the user is spared having to deal with loose tubing extending across the body, arms and hands as is common with existing airflow respiration monitoring devices having a control and sensing component supported off of and away from the body. Moreover, by extending the tubular interconnect line 96 and the remote sensor component 94 directly from the shoulder position to the nose of the user, disturbance of the correct nasal position by the remote sensor component 94 (in this case the nasal cannula and sensor) is less likely.

In the example shown, the airflow respiration monitoring device 90 is supported on the stiffening member 50, with the tubular interconnect line 96 secured along the shoulder attachment member 18. As such, a position of the airflow respiration monitoring device 90 relative to the user can be stabilized during use due to the stable platform provided by the back-mounted support device 10.

As another example of the back-mounted support device 10 providing a stable platform for structurally positioning various monitoring devices along the stiffening member 50, the shoulder attachment members 16 and 18, and/or the waist attachment members 26, 28, and relative to the body of the user, the back-mounted support device 10 can further comprise a remote tension sensor component (RISC) (or other type of respiration sensor) 102 comprising a tension sensor 104 coupled to at least one respiration band 106 which can be applied in tension around the user's chest and/or abdomen for monitoring respiration, and particularly to detect and measure displacement or expansion of the chest or abdomen of the user due to breathing. In this particular example, the RISC 102 can be used to monitor respiration and expansion of a thoracic or chest region of the user. The respiration band 106 can be coupled to the tension sensor 104, and the tension sensor 104 mounted to the stiffening member 50 at a mounting location 108 to provide a stable anchor point for the respiration band 106 with respect to the thoracic region of the user. The tension sensor 104 can be mounted to or otherwise supported by the stiffening member 50 using any known technique as described herein with respect to attaching or mounting or otherwise securing monitoring devices to the back-mounted support device 10.

The respiration band 106 can comprise any type of respiration band. In one example, the respiration band 106 can comprise an elastic type of band that permits a degree of expansion or stretch of the respiration band 106 along a longitudinal axis. The respiration band 106 can have ends configured to separably join to one another (e.g., via a buckle 110 or other connecting hardware) to facilitate easy application of the respiration strap 106 around the chest or abdomen of the user. Moreover, the respiration band 106 can have an adjustment member (e.g., a strap slider adjuster or other adjustment hardware) to facilitate adjustment of the length of the respiration band 106 so as to accommodate different sizes of users. Tension is maintained against the user's body sufficient to register any movement that can arise from the expansion and retraction of the respiration band 106. Such movement can be detected by, and the tension sensor 104 can comprise, any number of sensor types and/or configurations, such as a piezo type of sensing component capable of generating a voltage with the applied tension.

The back-mounted support device 10 is shown as further comprising a second remote tension sensor component (RTSC) (or other type of respiration sensor) 112 comprising a tension sensor 114 coupled to a respiration band 116 which can be applied in tension around the user's chest and/or abdomen for monitoring respiration expansion of the user. In this particular example, the second RTSC 112 can be used to monitor respiration and expansion of an abdominal region of the user. The respiration band 116 can be mounted to the stiffening member 50 at a mounting location 108 to provide a stable anchor point for the respiration band 116 with respect to the user. Providing first and second RTSCs (or other types of respiration sensors) provides the advantage of measuring the expansion of both the thoracic and abdominal regions at the same time via the stable platform provided by the back-mounted support device 10.

The back-mounted support device 10 provides a unique sensor configuration for measuring and monitoring thoracic and/or abdominal respiration parameters, including respiration rates. One advantage of this is that the one or more respiration type of monitoring devices can be utilized with another type of monitoring device also supported on the back-mounted support device 10, such as one or more position sensors. In this example, data from these different types of monitoring devices can be obtained and various correlations made, such as matching body positions of the user during sleep with the occurrence of one or more apnea events of interrupted breathing or other conditions of importance. A medical assessment can thereby be made directly comparing the type of body position that existed at the time of the apnea event. This can be useful for both medical evaluation as well as future position modification use to avoid problematic body orientations of the user.

Although specific types of monitoring devices are supported on the back-mounted support device 10 are discussed above, it is noted herein that these are not intended to be limiting in any way. Indeed, it is contemplated herein, and it will be apparent to those skilled in the art, that the back-mounted support device 10 can be sized and configured to support a number of different types of monitoring devices although not specifically mentioned or discussed in detail herein, such as a smart watch type of sensor 120 shown supported on the waist attachment member 28 (in phantom lines).

The monitoring devices can be mounted, coupled or otherwise supported on or by the back-mounted support device 10 in a variety of ways and using a variety of different attachment or supporting methods. Different monitoring device types may require different attachment or supporting means and methods. In addition, the particular component of the back-mounted support device 10 to which a monitoring device is attached may require a particular type of attachment. In any event, several different types of attachment means and/or methods are contemplated herein, which can include, but are not limited to, operable and closeable pouches, pockets, sleeves formed in the back-mounted support device 10 that facilitate the removable attachment of the monitoring devices (e.g., using zippers, snaps, buttons, and others), clips, pins, hook and loop fasteners, adhesives, and any combination of these. Those skilled in the art will recognize that other attachment means and methods can be employed to support one or more monitoring devices on the components of the back-mounted support device 10 that are not specifically mentioned herein.

The back-mounted support device 10 can further comprise one or more position control devices. Position control devices can comprise one or more objects or devices that are a fixed component of the back-mounted support device 10, or one or more objects or devices that are removably attached to one or more other components of the back-mounted support device 10, and that function to limit and/or control the bodily movements or orientation of the user, to prohibit certain bodily movement, positions or orientations of the user, etc. Position control devices can include, but are not limited to, back pillows, back cushions, back rails, back balls, and electronic or mechanical sensors that provide vibrator or minor shock alerts of occurrence of a predetermined position, and others.

As an example of the back-mounted support device 10 providing a stable platform for a position control device supported by or about the stiffening member 50, the shoulder attachments 16, 18, and/or the waist attachment members 26, 28 and proximate the user's back, the back-mounted support device 10 can further comprise a position control device in the form of a back pillow 130 (shown in phantom lines in FIG. 1A as an optional device that can be removably attached to the back-mounted support device 10, and as an attached component of the back-mounted support device 10 in FIG. 1B) supported on or proximate the stiffening member 50 via upper and lower pillow attachment systems 140, 148.

Applicable to this example, and as will be discussed in greater detail below, the back-mounted support device 10 can further comprise a system or device (e.g., any suitable means) for attaching the back pillow 130 to or in a location proximate the stiffening member 50, such that the back pillow 130 and the upper and lower pillow attachment systems 140, 148 are stabilized by the stiffening member 50. The stable platform and stabilizing function provided by the stiffening member 50 to the back pillow 130 and the upper and lower pillow attachment systems 140, 148 is discussed below. In one aspect, the back-mounted. support device 10 can comprise an upper pillow attachment system 140 and a lower pillow attachment system 148 at opposing ends of the stiffening member 50 for attaching the back pillow 130 at opposing top and bottom ends 54, 58 thereof (or proximate thereto), thereby configuring the back pillow 130 to further be positioned proximate the user's back and along the spine. Each of the upper and lower pillow attachment systems 140 and 148 can comprise a configuration that provides for free rotation and fall of the pillow between opposing positions along the user's back at right and left sides in response to gravity (discussed hereafter). in other words, the upper and lower pillow attachment systems 140 and 148 operate to provide a hinge type of coupling of the back pillow 130 to or proximate the stiffening member 50, wherein the back pillow 130 is rotatably coupled and configured to rotate in different directions about the stiffening member 50. As will be discussed further below, and with respect to any of the back-mounted support devices discussed herein, during use, with the user donning the back-mounted support device 10 with the back pillow 130 coupled thereto, and lying on one side in bed, the back pillow 130 operates to rotate to a first blocking position between the user's a right backside and a surface of a bed when the user moves to a right side sleeping position or orientation, which first blocking position functions to prohibit the user from unintentionally rolling over across his back to a supine position (i.e., a back sleeping position). By “unintentionally”, it is meant that the user attempts to move to this position unconsciously during sleep. However, the user can intentionally reposition to an opposite or left side sleeping position or orientation by rolling across his stomach to the opposite side if desired. Upon reaching the other side position, the back pillow 130 functions to rotate under the influence of gravity to a second control or blocking position between a left back side of the user and the surface of the bed to again function to prohibit the user from unintentionally rolling over across the back to a supine position (i.e., a back sleeping position). This feature enables a user to control body position on the bed, particularly when a side sleeping orientation is desired.

Several issued United States patents have been granted relating to rotatable back pillows capable of attachment to a human body to restrain rotation to a supine position from right and left side sleeping orientations during sleep. For example, see U.S. Pat. Nos. 8,429,775; 8,720,447; 9,585,499 and 10,477,976, each of which are hereby incorporated by reference in their entirety herein and as background to the present disclosure.

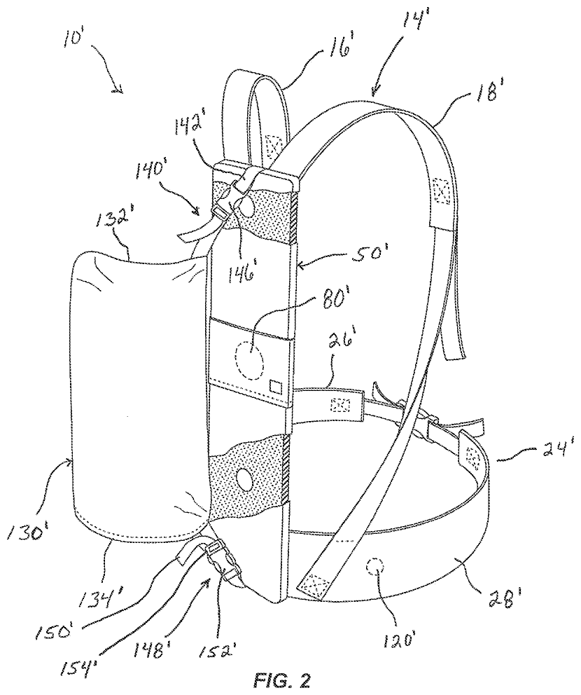

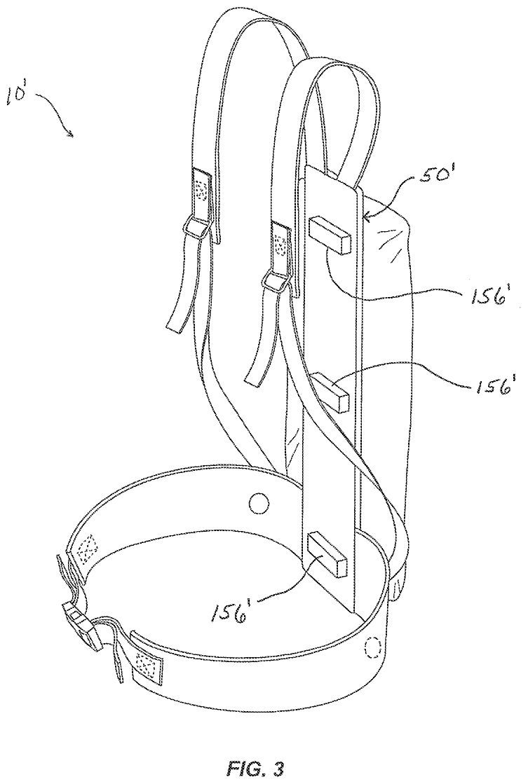

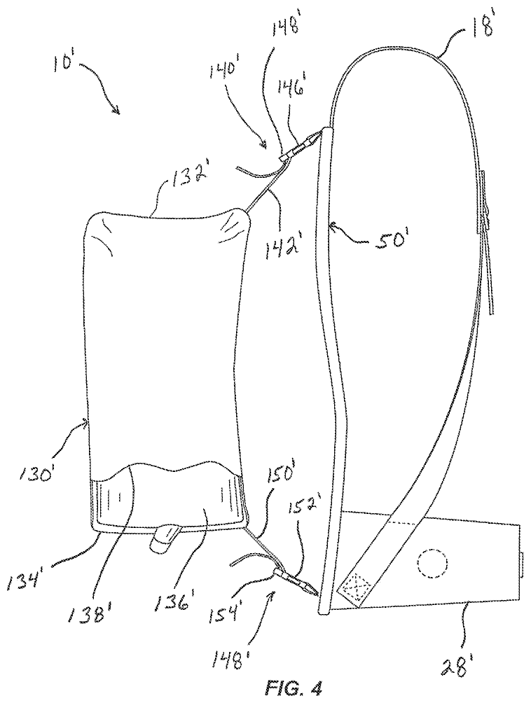

With reference to FIGS. 2-4, illustrated is a back-mounted support device 10′ in accordance with an example of the present disclosure. The back-mounted support device 10′ is similar in many respects, and can be configured similar to, the back-mounted support device 10 discussed above, and shown in FIGS. 1A-1B. As such, the description above can be referenced and incorporated here for support and understanding of the back-mounted support device 10′, where applicable as will be recognized by those skilled in the art. Indeed, the back-mounted support device 10′ is shown as being configured the same as the back-mounted support device 10 of FIGS. 1A-1B, except without some of the monitoring devices. As such, the back-mounted support device 10′ is shown as a harness-type device, comprising shoulder attachment members 16′, 18′, waist attachment members 26′, 28′ and a stiffening member 50′ supported by these, with each of these being shown as being the same as the shoulder attachments 16, 18, waist attachments 26, 28 and a stiffening member 50 of the back-mounted support device 10 of FIGS. 1A-1B. The back-mounted support device 10′ can further comprise one or more position control devices, such as a back-pillow 130′, which is shown as being the same as the back pillow 130 of the back-mounted support device 10 of FIGS. 1A-1B, In this example, and as so configured, the back-mounted support device 10′ can be primarily used for the purpose of controlling body position and orientation of a user during sleep. Although configured without many of the monitoring devices of the back-mounted support device 10 of FIGS. 1A-1B, the back-mounted support device 10′ can optionally comprise one or more monitoring devices suitable for evaluating body position during a sleeping event (e.g., see monitoring devices 80′ and 120′ in the form of body position sensors), without necessarily comprising other types of sensors or monitoring devices.

For individuals that are sensitive to objects in contact with their back while trying to sleep, the back-mounted support device 10′ can further comprise one or more spacer elements 156′ (see FIG. 3) positioned at one or more locations on the stiffening member 50′ and oriented for interfacing contact at the user's back to thereby space the stiffening member 50′ out of direct contact with the user along a substantial length of the stiffening member 50′. The spacer elements 156′ can be configured to reduce potential discomfort as otherwise might be experienced from direct contact of the stiffening member 50′ with the user's back. The spacer elements 156′ can comprise any material or composition of materials capable of facilitating a comfortable interface at the user's back. In one example, the spacer elements 156′ can comprise a compliant or elastically deformable material operable to at least partially compress under a load as applied by the stiffening member 50′ at the user's back. Example compliant or elastically deformable materials can include, but are not limited to, foam, soft rubber, and others. The spacer elements 156′ can comprise the same or different sizes, and can be spaced apart at different locations along the stiffening member 50′. The spacer elements 156′ can be supported on the stiffening member 50′ using a variety of means. In one example, the spacer elements 156′ can be attached to the stiffening member 50′ using adhesives, straps, hook and loop fasteners, snaps, and others. The spacer elements 156′ can be permanently fixed, or removably attached to the stiffening member 50′. In the example shown, the back-mounted support device 10′ comprises three foam spacer elements 156′ evenly spaced from one another and adhered to the stiffening member 50′. Other types of spacer elements are contemplated that could alternatively be used to provide air space between the stiffening member 50′ and the user's back, such a thin corrugated facing or mesh coupled to the stiffening member 50′, the facing or mesh having open spaces for air movement between the facing or mesh and the user's back.

A description with reference to FIGS. 1-5 will now be given with the understanding that any reference to a numbered element shown in FIGS. 1A-1B (e.g., the back-mounted support device 10) will also include a reference to the same or like element shown in FIGS. 2-4 (e.g., the back-mounted support device 10″), and vice versa, these being the same as indicated above (except where noted otherwise), and for purposes of discussion herein.

Generally speaking, side sleep positions are often recommended for individuals that are vulnerable to unhealthy conditions resulting when the body sleeps in a supine or back sleeping orientation or position. Positional sleep apnea and snoring are examples of such problematic health concerns. Side sleeping is now a medically recognized adjunct therapy for positional sleep apnea. One of the primary advantages of implementing a side sleeping therapy is a greatly reduced cost, compared to continuous positive airway pressure (CPAP) and other more invasive methodologies.

Numerous devices have been developed to encourage side sleep; however, many of these devices are uncomfortable to the user and therefore result in noncompliance or irregular usage. The present disclosure offers the desired blocking function against supine sleep; however, the unique comfort and effectiveness of the back-mounted support devices including a removable back pillow as discussed herein function to minimize numerous problems existing with prior art back pillows and back pillow systems. By minimizing discomfort and providing improved convenience and control of use, the back-mounted support devices (including a back pillow) enable a user to maintain long term, regular back pillow usage that may enable eventual training of the user's body to prefer and substantially sustain the desired side sleeping orientation versus rotating to a supine orientation, even with periodic nonuse of the back-mounted support device with the back pillow.

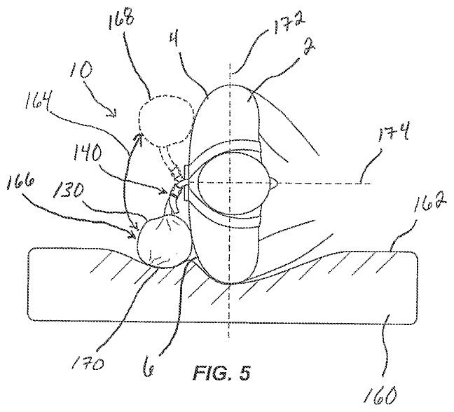

As indicated above, the back-mounted support device 10 can comprise a position control device in the form of a back pillow (e.g., back pillow 130). In the example shown, the back pillow 130 can comprise an elongate, tubular shaped pillow having a top end 132 and an opposing bottom end 134, each of which are attached to the stiffening member 50 or attached at a location proximate the stiffening member 50 (e.g., at a location on one or both of the shoulder attachment members 16, 18 and one or both of the waist attachment members 26, 28), so as to position the back pillow 130 relative to the user's back and along the user's spine. In one aspect, the back pillow 130 can comprise a light weight, inflatable insert 132 positioned within a suitable sleeve 134 having a frictional surface configured to interface with a surface of a bed (e.g., see bed 160 in FIG. 5), and which is capable of resisting sliding movement relative to the surface of the bed 160, which may include any linens disposed on the bed 160 (e.g., sheet, blanket, etc.). If significant enough, sliding movement of the back pillow 130 can defeat the captured position of the back pillow 130 between the user and the bed 160 needed to block rotation of the body. Light weight, elongate, inflatable pillow configurations are desired with a uniform radius along a longitudinal axis to maximize contact surface with the bed 160. The back pillow 130 can comprise a radius within the range of approximately 2 to 8 inches. However, for very large and heavy individuals, this could be greater. Guidelines for pillow construction are found in the referenced patents identified above, and are not discussed in detail herein.

Use of an inflatable insert 136 provides a cushioned comfort during use as well as minimum heat accumulation from body temperature. This may be realized from, and the inflatable insert 136 can be formed from, a variety of materials including, but not limited to, neoprene, silicone rubber, natural gum rubber, santoprene rubber, polyvinylchloride (PVC), latex, polyesters, nylons, and various polyesters having various types of laminates such as thermal polyester laminates. The sleeve 138 can be comprised of a soft fabric or other material that is comfortable to touch and that provides a surface to bed interface having a coefficient of friction sufficient to maintain the back pillow 130 in a control or blocking position between the user's back/side and the bed surface, and to resist displacement from the blocking position under pressure. Stated differently, the sleeve 138 of the back pillow 130 can be configured to comprise a frictional surface that, when interfaced with the bed surface, and with the back pillow 130 in the control or blocking position, resists sliding along the bed surface, particularly at times when the user attempts to roll towards a. supine position from the side sleeping position. In one example, not to be limiting in any way, the sleeve 138 can be made of flannel. However, other soft materials are contemplated.

The top end 132 or other upper portion of the elongate back pillow 130 can be coupled to or proximate (in some examples removably) the top end 54 of the stiffening member 50 via an upper pillow attachment system 140. The upper pillow attachment system 140 can comprise any type of system configured to couple (and in some examples removably couple) the upper portion of the back pillow 130 to or proximate the stiffening member 50 (and optionally to facilitate adjustment of the position of the back pillow 00 relative to the stiffening member 50). The upper pillow attachment system 140 can comprise a length which provides for free rotation of the back pillow 130 between opposing positions along the user's back at right and left sides in response to gravity. In one example, the upper pillow attachment system 140 can comprise a narrow, flexible strap or cord 142 coupled to and between the top end 132 of the back pillow 130 and the stiffening member 50. The strap or cord 142. can comprise first and second segments that connect together, such as via a buckle 146, to facilitate selective connection and disconnection of the back pillow 130 to/from the remainder of the back-mounted support device 10. The upper pillow attachment system 140 can further comprise a moveable clip, slide or clasp 148 coupled to the strap or cord 142 (or which can be part of the buckle 146) between the back pillow 130 and the stiffening member 50 and provides one example of a suitable adjustment member for selecting the proper length of the upper pillow attachment system 140.

The bottom end 134 of the elongate back pillow 130 can be coupled to or proximate (in some examples removably) the bottom end 58 of the stiffening member 50 via a lower pillow attachment system 148. The lower pillow attachment system 148 can comprise any type of system the same as or similar to the upper pillow attachment system 140. The lower pillow attachment system 148 can comprise a length which also provides for free rotation of the back pillow 130 between opposing positions along the user's back at right and left sides in response to gravity. In one example, the lower pillow attachment system 148 can comprise a narrow, flexible strap or cord 150 coupled to and between the bottom end 134 of the back pillow 130 and the stiffening member 50. The strap or cord 150 can comprise first and second segments that connect together, such as via a buckle 152, to facilitate selective connection and disconnection of the back pillow 130 to/from the remainder of the back-mounted support device 10. The lower pillow attachment system 148 can further comprise a moveable clip, slider or clasp 154 coupled to the strap or cord 150 (or which can be part of the buckle 152) between the back pillow 130 and the stiffening member 50 and provides one example of a suitable adjustment member for selecting the proper length of the lower pillow attachment system 148. The upper and lower pillow attachment systems 140, 148 can have the same or different attachment lengths, which allow the back pillow 130 to be properly spaced from the stiffening member 50 along the length of the back pillow 130, and which provide for a proper rotational radius and alignment of the back pillow 130 in the opposing control or blocking positions along the user's back at right and left sides upon rotation of the back pillow 130 between the opposing control or blocking positions. The adjustable positioning of the pillow allows the user to personalize placement relative to the user's body shape and unique sensitivities to pressure and functionality. Indeed, the respective upper and lower pillow attachment systems 140, 148 can be selectively adjustable to enable changing their lengths to meet the multiple requirements or desires of proper positioning and rotational radius of the back pillow 130 to achieve ideal opposing blocking or control positions for differing body sizes. These opposing control or blocking positions operate to provide the desired blocking function between the attached back pillow 130 and the bed surface 162 to thereby prevent the user from rotating from the desired side-sleeping orientation to a supine position. A variety of pillow sizes may also be substituted to properly fit the user's stature and weight.

As shown, a typical user 2 can be positioned on a bed surface 162 in a desired right side-sleeping orientation, represented by the vertical axis 172, in contrast to a supine orientation represented by the horizontal axis 174. Because of the stability of the stiffening member 50, the lengths of the upper and lower pillow attachment systems 140, 148 can be made adjustable in a continuous manner. This enables the back pillow 130 to have a specific swinging path following a rotational path 164 from the left side 4 of the user to the right side 6, corresponding to the illustrated back pillow 130 blocking position 166. Typically the user will roll across the front side of the body to shift sides to the left side-sleeping position. This rotation automatically initiates a gravity induced or activated fall of the back pillow 130 to the opposing control or blocking position 168 (illustrated in phantom lines) at the user's left, backside 4 with the user in a left side sleeping position (not shown). Any attempted rotation by the user from this side sleeping position to the supine position will function to capture the back pillow 130 between the user and the bed.

This rotational path 164 of the back pillow 130 ideally needs to be carefully set for the particular user and the user's bed 160 at home or in a care center. It is noted that rotational radius provides a stable reciprocating rotation of the back pillow 130 from side to side, enabling it to fall to alternating proper blocking positions, captured between the user's back sides 4 and 6 and the bed surface 162. More precisely, achieving the preferred back pillow capture involves coordination of the proper back pillow contact sites on the individual's back side with the respective mattress locations, to capture the back pillow 130 in early blocking contact at the very initiation of rotation. This effectively prevents the user from progressing past the commencement of rotation into a full rotation sequence where the user's momentum will actually accelerate continued rotation due to body weight.

Accordingly, the blocking contact preferably arises early in the rotation, thus impeding any continued progress toward full rotation. As soon as the user starts to rotate, the back pillow 130 is intended to be caught in the captured position, which is immediately registered in the user's mind as a blocking impediment. Over an extended period of repeated use, the repeated slight contact will automatically discourage further initiation of rotation toward the supine position. In other words, by impeding the initial minimal rotational movement, the potential for subsequent greater rotational momentum is minimized. The early blocking action literally biases the user to return to the desired side orientation with very little effort or attention, discomfort or interruption of sleep. In contrast, if the user is allowed to partially fall further toward the supine position, significantly greater effort must be exerted to return to the vertical orientation. This can result in arousal from sleep and unnecessary discomfort.

The difficulty of realizing a proper captured position of the back pillow 130 between the user and the bed surface arises because different users can have a different body size, physic and weight. In addition, the back pillow 130 can be capable of a number of different inflation amounts that can vary the stiffness of the back pillow 130, such as may be desired by the user. These varying parameters illustrate the importance of the adjustability of the upper and lower pillow attachment systems 140, 148 in achieving a proper offset positon and rotational radius of the back pillow 130 relative to the stiffening member 50, as well as proper body contact locations. In addition to these variables, further or fine tune adjustments to the upper and lower pillow attachment systems 140, 148 may be needed to achieve a correct swing or rotational radius. For example, additional adjustment or sizing may be needed on site because bed mattresses 50 can have significantly varying degrees of softness resulting in varying degrees of mattress depression due to different body weights and sizes of different users. As the back pillow 130 is loaded under partial capture at the user's side, the back pillow 130 is caused to be depressed (see item 170) into the mattress according to the mattress softness. The depression by the back pillow 130 directly modifies the captured position and attachment length with the user's back location. This is in addition to central mattress deflection along axis 172, as shown, which also modifies the captured position. Although it has previously been very difficult to precisely predict a correct swing or rotational radius for the back pillow 130 because of the unique body weight and. dimensions of each user, the mattress softness, and the degree of inflation of the pillow, the back-mounted support device 10 of the present disclosure, with its adjustable upper and lower pillow attachment systems 140, 148, helps to minimize the difficulty of these issues, comparatively speaking, wherein the upper and lower pillow attachment systems 140, 148 facilitate efficient and accurate positioning of the back pillow 130 to achieve the correct rotational radius across multiple different users.

An equally satisfactory method for selecting the desired rotational radius of the back pillow 130 is to adjust the lengths of the upper and lower pillow attachment systems 140 and 148 with the user in the side sleeping position on the bed surface 162 so that the back pillow 130 is actually positioned at the blocking location relative to the user lying on the bed surface 162. With the pillow positioned at the blocking location with respect to the user, the straps 112 and 150 of the upper and lower pillow attachment systems 140 and 148 can then be adjusted to a correct length. Although this may not expressly result in the “captured” position as described above, the blocking position will function to impede supine sleeping. This method has the advantage of being a. simple technique that can be accomplished by an attendant capable of selecting the proper back pillow 130 position and length of the respective straps 142, 150 with the user in the actual side orientation on the bed 160.

Earlier efforts by the inventor to meet this blocking or capture requirement have been attempted using a variety of shirt and vest configurations and sizes that have made it difficult to develop a “one size fits all” configuration. The inability to precisely define the correct shirt or vest size results in the occurrence of slack or a loose fit around the trunk of the body of the user when the garment is not a stretch, snug-fitting style. When there is excessive slack in the garment surrounding the user's chest, the variation in slack allows the garment itself to migrate around the body as the user moves in bed. This translates directly into instability in positioning the back pillow and consequently the “capture” or blocking site of the back pillow on the mattress is compromised. Accordingly, the desired result of both comfort and effective blocking action has been very difficult, and often frustrating to the user and supplier. The benefit of the stable stiffening member 50 and the variable length adjustment of the upper and lower pillow attachment systems 140 and 148 of the back-mounted support device 10 set forth herein are significant structural enhancements that facilitate a more accurate sizing of the back pillow 130 and achievement of the blocking or capture points on bodies of differing size and weight. These factors also facilitate the realization of an otherwise elusive “one-size-fits-all” product.

As discussed above, the shoulder attachment members 16, 18 are coupled to the top end 54 of the stiffening member 50 and the waist attachment members 26, 28 are coupled to the bottom end 58 of the stiffening member 50, thus stabilizing the upper and lower portions of the stiffening member 50 in a position relative to the user's back and along the user's spine. In a specific example, the shoulder attachment members 16. 18, the waist attachment members 26, 28, and the stiffening member 50 can be configured such that the stiffening member 50 is stabilized between the user's shoulder blades. In this position, the stiffening member 50 functions to provide a stable platform for the back pillow 130 by stabilizing the upper and lower pillow attachment systems 140, 148 as coupled to or proximate the stiffening member 50. More specifically, the stiffening member 50 provides a stable platform on which the upper and lower pillow attachment systems 140, 148 can be supported, thus facilitating the rotating or hinge-like functionality of the back pillow 130, as well as the adjustability of the rotational radius of the back pillow 130, as discussed herein. Stabilization is provided in part by the stiffening member 50 functioning to maintain the upper and lower pillow attachment systems 140, 148 in a consistent spaced arrangement relative to one another as supported, and in a consistent position relative to the stiffening member 50 and the user's back. Essentially, the upper and lower pillow attachment systems 140, 148, and their attachment locations on or relative to the stiffening member 50, are able to be supported in a fixed or substantially fixed relationship relative to one another, and relative to the body of the user. Stabilization is further provided by the stiffening member 50 functioning to support the various tension forces induced in the stiffening member 50 by the back pillow 130, such as those induced as a result of movement of the back pillow 130 relative to the stiffening member 50 and the user's back. Again, the upper and lower pillow attachment systems 140, 148 are stabilized in a fixed or substantially fixed position relative to each other and the user's back. Thus, it can be said that the stiffening member 50 provides the upper and lower pillow attachment systems 140, 148 and the back pillow 130 with controlled, stabilized and consistently maintained positional and orientation reference points relative to the body of the user through operational movements and range of motion of the back pillow 130. These reference points can be modified by adjusting the upper and lower pillow attachment systems 140, 148, wherein the modified reference points will also be stabilized. The shoulder attachment members 16, 18 and the waist attachment members 26, 28 are cooperatively configured to stabilize the position of the stiffening member 50 along the spine so as to maintain the proper reference points of the upper and lower pillow attachment systems 110,118 and the back pillow 130 (and the monitoring devices) relative to the body of the user even during movements made by the user, such as movements that occur during sleep, while at the same time preserving automatic rotational displacement of the back pillow 130 to left and right side blocking positions at proper displacement lengths to minimize the user's body rotation to a supine sleeping orientation.

In some situations, the back-mourned support device 10 may shift upward on the body during sleep due to body shape or restless movement. If needed, vertical position stability can be maintained by use of one or more connecting clips (see clips in FIG. 8A) or other fasteners attached at the back side of the waist attachment members which can be interconnected with the clothing of the user, such as bottom pajamas or under clothing worn around the hips while sleeping. The attached clip and waist attachment member prevent the harness and attached pillow from migrating upward, despite various movements of the body across the bed. Accordingly, the correct positioning of the back pillow 130 can be maintained using restraint caused by the attached clothing.

The back-mounted support device 10 as adapted to monitoring both sleep orientation and body parameters as part of a patient data resource can be useful in a variety of settings, including for senior patients. Medical support for such patients can be necessary as part of an incentive program to minimize return of the patient to hospital or other care and to establish and preserve health improving conditions for the patient during recovery periods. Various data networks exist to provide ongoing oversight of patient conditions so that adverse indicators can be managed at an early stage, prior to further decline of health. The importance of patient monitoring during periods of sleep has been previously emphasized. The back-mounted support member with the optional back pillow is an ideal system for monitoring various parameters that are helpful to ensure progressive healing of the patient and to avoid falls and other conditions that would necessitate renewed medical attention in a hospital or care center. As previously indicated, occurrence of supine sleep can enhance patient vulnerability to sleep apnea, snoring and other forms of sleep disturbance. Resulting conditions of tiredness create increased risk for falls, as well as aggravation of other medical risk conditions.

With reference to FIGS. 6A-6B, illustrated is a back-mounted support device 310 in accordance with an example of the present disclosure. The back-mounted support device 310 is similar in many respects, and can be configured similar to, the back-mounted support device 10 discussed above, and shown in FIGS. 1A-1B. As such, the description above can be referenced and incorporated here for support and understanding of the back-mounted support device 310, where applicable as will be recognized by those skilled in the art. Indeed, the back-mounted support device 310 can be configured as a harness-type device, and can comprise shoulder attachment members 316 and 318, waist attachment members 326 and 328 and a stiffening member 350 supported by these to provide a stable platform for various monitoring devices. The back-mounted support device 310 can further comprise one or more position control devices, such as a back-pillow 430, wherein the back-mounted support device 310 also provides a stable platform for the position control device.

Unlike the back-mounted support device 10 discussed above and shown in FIGS. 1A-1B, the back-mounted support device 310 can comprise a stiffening member 350 having a rod-like configuration. Stated differently, rather than the stiffening member 350 comprising a flat, elongate configuration, the stiffening member 350 can comprise or be in the form of an elongate stiffening rod configured to be aligned with the spine of the user, and having a top end 354 a bottom end 358 and a spanning portion 360 extending between the top and bottom ends 354, 358. The elongate stiffening rod can have a high aspect ratio (e.g., a narrow cross-section (e.g., approximately less than half an inch in a measured cross-sectional dimension (e.g., a diameter compared with a length (e.g., 5-20 inches) of the elongate stiffening rod).

The stiffening member 350 in the form of the elongate stiffening rod (hereinafter stiffening rod 350) can be configured to comprise a width having a comparable unit dimension greater than that of a thickness of the stiffening rod 350 in order to inhibit deflection in one direction while enabling deflection in another direction along its length. In one example, the stiffening rod 350 can be configured to inhibit lateral deflection of the stiffening rod 350 about an axis normal (front to back) to the stiffening rod 350 and along its length, such as deflection laterally across a user's back when supported relative to the user's body, while at the same time enabling some degree of bending or flex or deflection in a direction about a lateral axis, such as deflection toward. or away from a user's body when supported relative to the user's body along the spine. This can obviously vary somewhat, depending upon body size and weight of the particular user. Such variations can be limited. to preserve the functionality (e.g., to provide stability) of the stiffening rod 350, while balancing comfort. In one aspect, the stiffening rod 350 can align with the spine of the user, and the length of the stiffening rod 350 can approximate the distance from the user's waist to just below the neck, wherein primary support of the back-mounted support device 310 can be applied at the user's waist (about the spine and/or the spine and the hip bones) for both stability and comfort.

As indicated, the stiffening rod 350 can be configured to be stiff in one direction, but flexible in another direction. In this example, the stiffening rod 350 can be configured to possess some degree of forward/rearward flexibility for enhanced comfort. Providing the desired stiffness can be achieved, at least in part, through various geometrical configurations of the stiffening rod 350, rather than through material selection alone where an appropriate modulus of elasticity can be a larger factor for consideration.

Moreover, the stiffening rod 350 can be configured to resist rotational twisting along its longitudinal axis. More specifically, the stiffening rod 350 can be configured such that the top end 354 and the bottom end 358 do not rotate or twist relative to one another along the longitudinal axis, thus maintaining these in a common plane. Therefore, any monitoring devices attached or mounted to the stiffening rod 350 at the upper and lower portions will also be maintained in the same plane. This is unlike the stiffening member 50 described in FIGS. 1A-1B in which the stiffening member 50 is configured to twist along its longitudinal axis, such that the surface of the stiffening member 50 at the upper and lower portions can be positioned in different planes relative to one another.

The back-mounted support device 310 can comprise a shoulder platform 320 supported by the shoulder attachment members 316, 318 to receive and facilitate the secure coupling of an upper portion 352 of the stiffening rod 350 to the shoulder platform 320. The shoulder platform 320 can be integrally formed with and a part of the shoulder attachment members 316, 318, or it can comprise a separate structure coupled to the shoulder attachment members 316, 318. The back-mounted support device 310 can further comprise a waist platform 330 supported by the waist attachment members 326, 328 to receive and to facilitate the secure coupling of a lower portion 356 of the stiffening rod 350 to the waist platform 330. The waist platform 330 can be integrally formed with and part of the waist attachment members 326, 328, or it can comprise a separate structure coupled to the waist attachment members 326, 328. In the example shown, the shoulder platform 320 is integrally formed with the shoulder attachment members 316, 318. Likewise, the waist platform 330 is integrally formed with the waist attachment members 326, 328. The shoulder platform 320 can be centrally positioned between the shoulder attachment member 316 and the shoulder attachment member 318, such that the shoulder platform 320 is positioned between the shoulder blades of the user when the back-mounted support device 310 is in use. The waist platform 330 can be centrally positioned between the waist attachment member 326 and the waist attachment member 328, such that the waist platform 330 is positioned at the lower back of the user.

The stiffening rod 350 can be supported between the shoulder platform 320 and the waist platform 330. More specifically, the upper portion 352 and top end 354 of the stiffening rod 350 can be coupled to the shoulder platform 320, and the lower portion 356 and bottom end 358 of the stiffening rod 350 can be coupled to the waist platform 330, wherein the stiffening rod 350 is configured to span or extend between these, thus connecting the shoulder attachment members 316, 318 to the waist attachment members 326, 328, wherein these operate to provide a stable platform of the back-mounted support device 310 similar to that described above. It is intended that the upper and lower portions of the stiffening rod 350 be securely coupled to the shoulder and waist platforms 320 and 330 so there is no relative movement of the stiffening rod 350 relative to the shoulder and waist platforms 320 and 330. With the stiffening rod 350 securely coupled to the shoulder and waist platforms 320 and 330, the shoulder and waist platforms 320 and 330 can be maintained in the same plane by virtue of the secure coupling to the stiffening rod 350. In other words, unlike the shoulder attachment members 16, 18 and the waist attachment members 26, 28 of FIGS. 1A and 1B that can twist out of plane relative to one another (e.g., as a user rotates his/her shoulders relative to his/her hips, such as during sleep), the shoulder and waist platforms 320, 330 shown here are maintained within the same plane and are not allowed to rotate (i.e., twist) relative to one another along a longitudinal axis of the stiffening rod 350 due to the stiffening rod 350 being securely coupled to these, and to the inherent resistance of the stiffening rod 350 to such rotation. Indeed, the stiffening rod 350 functions to inhibit rotation of the shoulder and waist platforms 320, 330 relative to one another. Therefore, any monitoring devices (e.g., body position sensors) supported on the shoulder and waist platforms 320, 330, or on the stiffening rod 350 at the shoulder and waist platforms 320, 330, that are positioned in the same plane, will also be substantially maintained within the same plane so as to provide substantially the same measured outputs. In this example, both the shoulder and waist platforms 320, 330, the stiffening rod 350, and any monitoring devices mounted thereto or otherwise supported thereon, can be positioned to lie substantially within the same plane, and to rotate together (such as when the user rotates from a side sleeping position to a supine position).

To further stabilize the stiffening rod 350 relative to the body of the user, and to further stabilize the combination of the stiffening rod 350, the shoulder attachment members 316, 318, and the waist attachment members 326, 328, relative to one another and the body of the user, the stiffening rod 350 can further comprise an upper transverse member 361 at the top end 354 of the stiffening rod 350, and a lower transverse member 363 at the bottom end 358 of the stiffening rod 350. Each of these can extend outward in opposing lateral directions any suitable distance relative to a longitudinal axis of the stiffening rod 350, and each can be configured to interface with the shoulder and waist platforms 320, 330, respectively, to further secure the stiffening rod 350 within these, and against undesired rotation or movement, such as rotation (twisting) along a longitudinal axis of the stiffening rod 350. The upper and lower transverse members 361, 363 function to increase the interfacing surface area between the stiffening rod 350 and the shoulder and waist platforms 320, 330, thus inhibiting rotation of the stiffening rod 350 relative to the shoulder and waist platforms 320, 330. The upper and lower transverse members 361, 363 also function to increase the rigidity of the shoulder and waist platforms 320, 330. In doing so, the shoulder and waist platforms 320, 330 and the stiffening rod 350 operate to model a rigid, unitary system, as much as possible.

The upper and lower transverse members 361, 363 can comprise various sizes and configurations. In one aspect, the upper and lower transverse members 361, 363 can comprise a similar cross-sectional area and configuration as the spanning portion 360. in another aspect, the upper and lower transverse members 361, 363 can comprise a flat, planar configuration operable to interface with and seat against respective surfaces of the shoulder and waist platforms 320, 330, and to provide a mounting surface capable of receiving and supporting one or more monitoring devices thereon. The stiffening rod can alternatively be rotationally stabilized by other cross members or stiffening designs in place of the transverse members that also function to define a stable sensor plane for the stiffening rod with respect to the user's back and to resist rotational twisting along a. longitudinal axis of the stiffening rod and movement away from the user's back, such as during sleep.

The top and bottom ends 354, 358 and the upper and lower portions 352, 356 of the stiffening rod 350 can be securely supported on the shoulder and waist platforms 320, 330 in a variety of ways. In one example, the top and bottom ends 354, 358 can be removably coupled to the shoulder and waist platforms 320, 330, respectively, using hook and loop fastening systems. In another aspect, as shown, the shoulder and waist platforms 320, 330 can each comprise an operable and closeable pocket or compartment (e.g., see zippered compartments 480, 482) sized and configured to receive and releasably retain the top and bottom ends 354, 358 of the stiffening rod 350. These two exemplary coupling means illustrate that the stiffening rod 350 can be removably coupleable to the shoulder and waist platforms 320, 330. As such, different sized stiffening rods (e.g., stiffening rods of different lengths) can be interchanged with one another depending upon the size of the intended user of the back-mounted support device 310. Moreover, the coupling means can function to secure the ends of the stiffening rod 350 to the shoulder and waist platforms 320, 330 in a manner so as to prevent, as much as possible, any movement between these, thus allowing the stiffening rod 350 and the shoulder and waist platforms 320, 330 to essentially model a rigid system, and to be stabilized substantially within the same plane (rotation of these relative to one another along a longitudinal axis of the stiffening rod 350 is resisted).

The back-mounted support device 310 can further comprise one or more stiffening inserts 488 and 490 (also referred to as sensor mounting bases in the event they support a monitoring device thereon) configured to interface with the upper and lower portions of the stiffening rod 350, respectively, and to help securely retain the stiffening rod 350 in a stable position as supported on the shoulder and waist platforms 320, 330, As in the example shown, the stiffening insert 488 can be caused to provide a stiffening interface between the upper portion 352 of the stiffening rod 350, including any upper transverse member 361, and the closeable compartment 480. The stiffening insert 488 can be sized and configured to substantially fill the volume of the closeable compartment, with a cutout (in this case a T-shaped cutout) sized and configured to firmly receive and support the transverse member 361 and possibly the upper portion 352 and upper end 354 of the stiffening rod 350. These can be secured within the stiffening insert 488 with an adhesive of other means to provide rigidity to the combined assembly. When the stiffening rod 350 is properly secured to the stiffening insert 488, these essentially model a unitary, rigid structure. The stiffening insert 488 can be made of any material that is stiff and that does not rotate about an axis. For example, the stiffening insert 488 can be made of a stiff foam or other material. A similar stiffening insert 490 can be configured to provide a stiffening interface between the lower transverse member 363 and the closeable compartment 482.

One benefit of the combination stiffening rod and stiffening insert configuration is the ability to connect a flexible shoulder attachment member with a flexible waist attachment member using the combined stiffening rod and stiffening insert to effectively cause a portion of these to function as a stiff component for the purposes discussed above, such as to maintain a stable positional orientation of the various monitoring devices. For example, the position sensors can maintain a related and known orientation with respect to the body to determine sleep issues that arise based on body position. Monitoring devices, such as respiration bands and other sensors can have a fixed point of attachment to the stiffening rod (and/or to the combination stiffening rod and stiffening insert) to maintain a stable position and reference point, avoiding a common problem of shifting positions on the body for respiration bands attached around the chest or abdomen. In short, the use of a spine-oriented. stiffening rod between the shoulders and waist of the user offer significant solutions to long standing problems in this general field of medicine.

The rotational stability of the stiffening rod 350 allows the stiffening rod 350 to support a sensor mounting base 492 comprising a planar platform operable to receive and support a monitoring device, such as a body position sensor 493. The sensor mounting base 492 can include a stiff or rigid configuration. The sensor mounting base 492 can be fixed to the spanning portion 360 of the stiffening rod 350, as shown, so as to be positioned within a plane common with that of the upper and lower portions of the stiffening rod 350 (and/or the combination stiffening rod and stiffening insert). Thus, a monitoring device supported thereon can be positioned and oriented in the same plane as a monitoring device supported on the upper and/or lower portions of the stiffening rod (and/or the combination stiffening rod and stiffening insert).

In light of the above discussion, it is noted that the stiffening rod 350, and/or the shoulder and waist platforms 320, 330 supporting the combined stiffening rod and stiffening insert assembly, provides ideal locations for attaching one or more types of monitoring devices, such as one or more body position sensors configured to monitor body movement and orientation of the body during sleep. Although the back-mounted support device 310 does not provide the same twisting rotation as the back-mounted support device 10 discussed in reference to FIGS. 1A-1B, the back-mounted support device 310 still provides a stable platform for the monitoring devices in that it provides a stable position and orientation of the monitoring devices relative to the body of the user, where the correlated reference points of the monitoring devices with the desired positions relative to the body of the user are maintained, even through various movements by the user. Further discussion of the stable platform functionality provided by the back-mounted support device 310 is not provided as the discussion of providing a stable platform detailed above with reference to FIGS. 1A-1C is applicable to all of the back-mounted support devices disclosed herein.

The stiffening rod 350 may be constructed of various lightweight materials that provide the desired rotational and translational stiffness, such as a fiber composite composition similar to those used to construct a dowel or fishing rod. Other materials can include, but are not limited to, other light weight plastics or metals. Again, the lateral width of the stiffening rod 350 may be greater that the forward thickness or diameter to restrict lateral deflection of the rod away from the spine alignment. In some examples, the upper portion of the stiffening rod 350 can be formed with a slight curve away from the user's body (see FIG. 8A) to follow the curvature of the user's back. A variety of curved configurations for the stiffening rod may be selected, conforming to the spinal configuration of the user. Indeed, the curvature of the stiffening rod may be precisely structured to conform to the spinal curvature of the user. This is particularly advantageous for older users that may have substantial spinal deformation. In another example, when using a tapered flexible fiber composite construction, the stiffening rod 350 can be limited to or can comprise a length slightly longer than a shorter spaced apart distance between the shoulder and waist platforms, such that when coupled to these, the stiffening rod 350 is caused to slightly flex in an outward direction away from the user's back wherein the stiffening rod 350 follows, at least in part, the curvature of the user's back. This is similar in function to a fishing pole bending into an arc with a reduced length.

The back-mounted support device 310 can further comprise one or more spacers (e.g., see spacers 456 and 457) supported on the stiffening rod 350 at the upper and lower portions of the stiffening rod 350), which function to interface with the user's back and to displace the stiffening rod 350 away from the user's back.

As an example of the back-mounted support device 310 providing a stable platform for structurally positioning various monitoring devices along the stiffening rod 350, the shoulder attachment members 316, 318, and/or the waist attachment members 326, 328, and relative to the body of the user, the back-mounted support device 310 can further comprise a remote tension sensor component (RISC) (or other type of respiration sensor) 412. The RTSC 412 is similar to the RTSC monitoring devices discussed above with respect to FIGS. 1A-1B in that its function is to measure monitor displacement or expansion of the chest or abdomen of the user due to breathing for the purpose of monitoring respiration. In this particular example, the RISC 412 can be used to monitor respiration and expansion of an abdominal region of the user. Unlike the RISC monitoring devices discussed above that are configured to wrap around the entire abdomen of the user using a continuous respiration band, the RTSC 412 operates to wrap partially around the user using two discontinuous respiration bands. As shown, the RTSC 412 can comprise a first tension sensor 414a (e.g., a transducer) coupled or mounted to the waist attachment member 326. The RTSC 412 can comprise a second tension sensor 414b similarly configured and coupled or mounted to the waist attachment member 328. The RISC 412 can further comprise a first respiration band 416a coupled to the first tension sensor 414a at one end, and, at an opposing end, to a stiffening rod mount 418 secured to the stiffening rod 350. The RISC 412 can further comprise a second respiration band 416b coupled to the second tension sensor 414a and to the stiffening rod mount 418. The first and second respiration bands 416a, 416b are thus configured to extend from a backside of the user in different directions relative to the stiffening rod 350 and to wrap around at least a part of the diaphragm and abdominal cavity of the user below the rib cage (along at least the lateral sides of the user) upon the user donning the back-mounted support device 310. The first and second tension sensors 414a, 414b can be mounted at any location on the waist attachment members 426, 428 to provide any desired wrapping distance of the first and second respiration bands 416a, 416b around the abdomen of the user. The first and second tension sensors 414a, 414b each are able to register breathing movement of the respective connected respiration bands 416a, 416b. The location of the respiration bands 416a, 416b at the sides of the user is less obtrusive than conventional abdominal band positions, and is ideal for support between the stiffening rod 350 at one end and the waist attachment member-mounted tension sensors 414a, 414b at the other end. Any distension of the diaphragm and abdomen will be detected to provide respiration rate in real time.

In another example, the stiffening rod mount 418 can be slidably coupled to the stiffening rod 350, such that the stiffening rod mount 418 can be adjusted to different positions along the stiffening rod 350 to accommodate users of different size. In this example, the first and second respiration bands 416a, 416b can also be slidably coupled to the first and second tension sensors 414a, 414b to facilitate adjustment of the stiffening rod mount 418, and to ensure that the first and second respiration bands 416a, 416b are able to establish a proper tension after an adjustment has occurred. As shown, the respiration bands 416a, 416b can each comprise a length sufficient to allow different parts of the respiration bands 416a, 416b to be moved into place to be secured to the tension sensors 414a, 414b to facilitate a variety of adjustment locations.

Similar to these described body parameters, blood oxygen levels, heart rate, blood pressure, temperature, incontinence, respiration, etc. are important body parameters that can be monitored using additional monitoring devices supported on the back-mounted support device 310 (or any of the back-mounted support devices discussed herein, such as back-mounted support devices 10 and 10′ of FIGS. 1A-1B, 2-4), As shown with respect to back-mounted support device 310, monitoring devices 502, 504, 506, etc., can be included in the back-mounted support device 310 for measuring such conditions, and can be mounted at various locations on the back-mounted support device 310, such as on the waist attachment members 316, 318, the shoulder attachment members 326, 328, and/or the stiffening rod 350. For example, body position sensors have been disclosed that may be represented by monitoring device 504, with its reference orientation in a vertical side-sleeping reference condition. The occurrence of supine position of the patient would be noted and could be correlated with other body parameters that normally accompany apnea events in the user, prompting required use of CPAP or positional therapy such as the subject back pillow. Monitoring device 502 could be used to provide or be operable with a moisture sensor 503 with remote extension for alerting incontinence events with the user. A temperature sensor 508 in contact with the patient skin can be used to provide temperature measurement. Blood pressure, heart rate, blood oxygen levels, and other internal parameters can be measured with existing sensors commonly used in watches and finger mounts, in which the sensor network is exposed to the skin of the user, such as through a window of a pocket in the waist attachment member 506 housing the sensor. All of the above monitoring device, and others, can be linked through wireless systems or through connecting wires 511, 513, 515. Telemetry metering can be accomplished through a single monitoring device 105 similar to a conventional smart watch element. Data flow can then be relayed to a central monitor for transmission to medical and other attending personnel. In addition, an LED light 510 for nighttime illumination may be coupled at the front of one or both of the shoulder attachment members to facilitate lighting of the area around the user, thus helping to minimize stumbling and falling by the user. The LED light 510 could be activated automatically when one or more position sensors register that the user is standing or has fallen from bed.

The back-mounted support structure 310 can further comprise one or more position control devices, such as a back pillow 420 operably coupled to or proximate the stiffening rod 350 via upper and lower pillow attachment systems 440, 448, such that the back pillow 420 and the upper and lower pillow attachment systems 440, 448 are stabilized by the stiffening rod 350. The back pillow 420 and the upper and lower pillow attachment systems 440, 448 can be configured and can function in a similar manner as the back pillow 130 and the associated upper and lower pillow attachment systems 140, 148 discussed above.

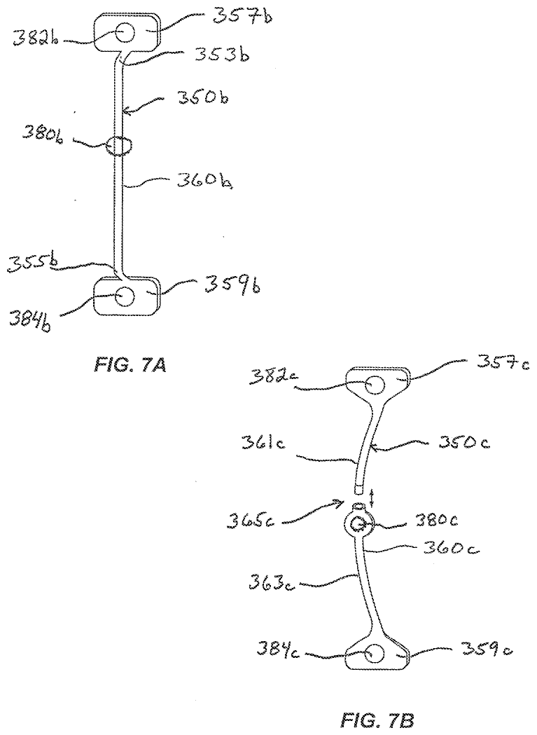

FIGS. 7A and 7B illustrate exemplary configurations of a stiffening rod, each of which can be incorporated for use into the back-mounted support device 310 of FIGS. 6A and 6B as an alternative to the stiffening rod 350. Specifically, FIG. 7A illustrates a stiffening rod 350b having a linear spanning portion 360b extending between top and bottom ends. The stiffening rod 350b can further comprise first and second elbow sections 353b, 355b located at the top and bottom ends, respectively, for elevating the spanning portion 360b away from the shoulder and waist platforms 320, 330 (see FIGS. 6A and 6B), as well as the back and spine of the user to avoid uncomfortable contact at the back. Rotational stability of the stiffening rod 350b is enhanced by mounting plates 357b and 359b supported by the first and second elbow sections 353b, 355b, respectively. The mounting plates 357b, 359b can be coupled to the respective shoulder and waist platforms 320, 330 in a similar manner as the transverse members of the stiffening rod 350 shown in FIGS. 6A and 6B to connect the stiffening rod 350b to the shoulder and waist attachment members 320, 330. The spanning portion 360b and the mounting plates 357b, 359b can further function to support one or more monitoring devices thereon, such as one or more body position sensors (e.g., see body position sensors 380b, 382b, 384b).

FIG. 7B illustrates a stiffening rod 350c having a curved spanning portion 363c extending between top and bottom ends. The spanning portion 363c is shown as comprising first and second segments 361c, 363c configured to be releasably coupled to one another via a connection interface 365c. The first and second segments 361c, 363c can comprise connecting ends defining the connection interface 365c. In one example, the first segment 361c can comprise a male configured connecting end operable to be received into a female configured connecting end of the second segment 363c and to be secured therein via a press or interference fit. The first and second segments 361c, 363c can comprise different lengths. Moreover, many segments of different lengths can be connected together, thus facilitating different full lengths of the stiffening rod 350c to adapt for users of different heights. The stiffening rod 350c can further comprise mounting plates 357c, 359c supported at the top and bottom ends that function similarly as the mounting plates discussed herein. The stiffening rod 350c can further comprise a mounting plate integrated into one of the first or second segments 361c or 363c, or both. The spanning portion 360c and the mounting plates 357c, 359c can further function to support one or more monitoring devices thereon, such as one or more body position sensors (e.g., see body position sensors 380c, 382c, 384c).

FIGS. 8A and 8B illustrate the back-mounted support device 310′ of FIGS. 6A and 6B donned by a user, with the back-mounted support device 310′ comprising an alternative shoulder attachment member configuration. As shown in this example, the back-mounted support device 310′ of FIGS. 6A and 6B can comprise a pair of shoulder attachment members 316′ and 318′ having distal ends 330′, 332′ that are coupled to the stiffening rod 350′ rather than to respective waist attachment members 326′, 328′. Specifically, the distal ends 330′ and 332′ can be coupled to a shoulder attachment member mount 335′ or any other type of mount or mounting system supported on the spanning portion 360′ of the stiffening rod 350′.

In another example, the shoulder attachment member mount 335′can be slidably supported on the stiffening rod 350′, thus facilitating adjustment of the attachment location of the shoulder attachment members 316′, 318′ along the spanning portion 360′ of the stiffening rod 350′. The shoulder attachment mount 335′ can be slidably or otherwise adjustably supported on the stiffening rod 350′ in a variety of ways. In one example, the shoulder attachment member mount 335′ can comprise a collar, and a set screw 337′ supported in the collar. The set screw 337′ can be threaded and supported within a threaded opening in the collar. The set screw 337′ can be caused to move annularly in a bi-directional manner. In one aspect, the set screw 337′ can be caused to move inward to engage a surface of the spanning portion 360′ of the stiffening rod 350′, thus locking the collar in the desired adjustment position to provide the desired attachment location for the shoulder attachment members 316′, 318′ about the stiffening rod 350′. If further adjustment is necessary or desired, the set screw 337′ can be moved outward to disengage the surface of the spanning portion 360′, wherein the slidable collar can he moved to a different position on the stiffening rod 350′. This configuration allows the shoulder attachment members 316′, 318′ to comprise any number of attachment locations.

With each of these examples, and with the back-mounted support device 310′ donned by a user, the attachment locations can be rearward of a mid-coronal plane. Moreover, such an attachment location can enhance the stability of the stiffening rod 350′ (i.e., enhance the stable platform of the back-mounted support device 310′) due to the direction of forces acting on the stiffening rod 350′ and against the body of the user by the shoulder attachment members 316, 318′ and the waist attachment members 326′, 328′ compared with the direction of forces resulting from an attachment location on the waist attachment members 326′, 328′ or one that is forward the mid-coronal plane. As discussed above, longitudinal forces acting along a longitudinal axis of the portion of the shoulder attachment members 316′, 318′ beneath the user's arms and passing through the mid-coronal plane along respective lateral sides of the user (which forces are induced by the attachment of the shoulder attachment members 316′, 318′ to the waist attachment members 326′, 328′ or the stiffening rod 350′ and the user donning the back-mounted support device 310′ as appropriately adjusted), can be caused to be applied in a direction transverse to the mid-coronal plane. Moreover, the relative distance that the shoulder attachment portions 316′, 318′ and the top portion or end 352′ of the stiffening rod 350′ will be drawn inward toward one another as a function of the distance that the stiffening rod 350 has a tendency to displace in a vertical direction upon the shoulder attachment members 316′, 318′ being adjusted and tightened against the user's body, will depend. upon the vertical attachment location of the distal ends 330′, 332′ of the shoulder attachment members 316′, 318′ to the stiffening rod 350′ along a vertical axis (normal to ground if the user were standing), and a measured distance between the attachment location for the distal ends of the shoulder attachment members 316′, 318′ and the attachment location of the proximal ends of the shoulder attachment members 316′, 318′ at the upper portion of the stiffening rod 350′. When the waist attachment members 326′, 328′ are suitably adjusted and their respective ends are secured to one another around the waist of the user, the resulting tension force generated within the waist attachment members 326′, 328′ operates to draw the bottom portion or bottom end 356′ of the stiffening rod 350 inward towards the user's back, With the shoulder attachment members 316′, 318′ and the waist attachment members 326′, 328′ so configured relative to the stiffening rod 350′ and the body of the user, the stiffening rod 350′ can be caused to be disposed in a more stable position relative to the user's back along the spine, while providing a greater resistance to subsequent displacement of the stiffening rod 350′ as a result of the more directionally inward forces induced within the back-mounted support device 310′ made possible by the attachment location for the distal end of the shoulder attachment members 316′, 318′ being rearward of the mid-coronal plane and attached to the stiffening rod 350.

In this example, the longitudinal forces acting along a longitudinal axis of the portion of the shoulder attachment members 316′, 318′ beneath the user's arms and passing through the min-coronal plane can be caused to be applied in a direction at an even greater transverse angle relative to the mid-coronal plane due to the distal ends of the shoulder attachment members being attached to the stiffening rod 350 at locations closer to the top end of the stiffening member 350′. In one specific example, the longitudinal forces can be caused to be applied in a direction perpendicular to the mid-coronal plane due to the shoulder attachment members being attached to the stiffening rod 350 at a location with the distal ends being positioned perpendicular to the stiffening rod 350. This can effectively eliminate any forces acting on the waist attachment members 326′, 328′ from the shoulder attachment members 316′, 318′ that would otherwise have a tendency to cause the waist attachment members 326′, 328′ to displace in a vertical direction upon the shoulder attachment members 316′, 318′ being adjusted and tightened against the user's body. Of course, different attachment locations are possible and contemplated herein. Moreover, as the shoulder attachment members 316′, 318′ operate to induce a force on the stiffening rod 350′ in a direction substantially perpendicular to the user's back that causes the stiffening rod 350′ to be drawn directly inward, thus pressing the shoulder and waist platforms 320′, 330′ against the user's back, and thus generating a more robust stable platform of the back-mounted support device 310. Stated differently, with this attachment location, the forces drawing the shoulder and waist platforms 320′, 330′ inward can all be normal and in a direction substantially perpendicular to the user's back, thus reducing or eliminating any inclined or angular components of these forces.

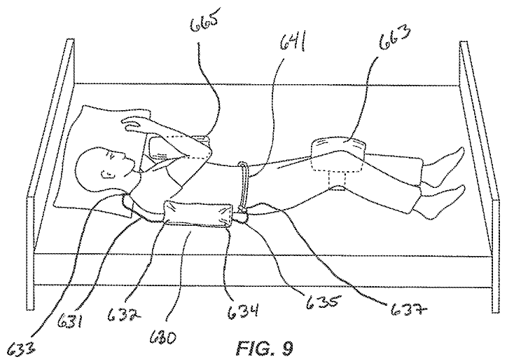

With reference to FIG. 9, illustrated is a back-mounted support structure [] comprising a decoy pillow 630 operable as a “decoy” to simulate the blocking function and attendant position control provided by any of the back pillows discussed above (e.g., see FIGS. 1A-1B, 2-4, 5A-5B). Unlike the back pillows discussed above, the decoy back pillow 630 comprises a comparative lessor level of functionality which can actually trigger a positional recovery response by a user to move back to and/or maintain a side sleeping position even upon slight contact with the decoy pillow 630 upon initiation of rotation toward a supine orientation. This lessor level of functionality distinguishes the decoy pillow 630 from the more functional back pillow configurations discussed above that are capable of actually physically blocking the user from completing rotation to the supine position upon encountering the back pillow in either of the described first and/or second control or blocking positions achieved by the back pillow rotating to these control or blocking positions when the user is in respective right and left side sleeping positions or orientations, as discussed above.

The decoy pillow 630 actually becomes functional for a user who has already been previously trained to favor side sleeping by prior repeated use of the actual blocking action by a rotatable back pillow (such as the back pillows 130, 430, 430′ discussed above) captured between a bed surface against one side of the back pillow and a user's left or right back side at an opposing side of the back pillow. As indicated above, such use and training is capable of providing the user with a “conditioned” awareness of an anticipated, actual blocking action by a back pillow which is fully capable of impeding rotation of the user's body to a supine position during sleep.

As used herein, the term “conditioned awareness” implies that the blocking action experienced by the user with the decoy pillow 630 triggers a physical and mental expectation that user's body will actually be blocked upon contacting the pillow. In this manner, initial contact with the decoy pillow 630 causes the user to instinctively recover to the side sleeping orientation without need of the decoy pillow 630 to actually fully block rotation. This allows the user to mentally interrupt further rotation of the body based on the expectation that further rotation would be unsuccessful and therefore pointless, immediately causing the user to return to the preferred side sleeping position.

Under the concept of preconditioned response by the user, the decoy pillow 630 is designed with a less robust body and reduced amount of blocking action to minimize the potentially intrusive nature of physically and aggressively blocking the ultimate rotation of the body to a supine position. Although not necessary, the decoy pillow 630 can be of smaller dimensions and weight compared to an actual blocking back pillow, such as those discussed above. The decoy pillow 630 can comprise an elongate flexible container, bag or sack without the need for a sleeve having substantial frictional surfacing, although such could be utilized. The decoy pillow 630 can be formed of a variety of materials, such as Mylar, rubber, latex, polychloroprene, various fabrics (e.g., nylon or other types of fabrics), rubber and others. Extensive and more prominent or pronounced shoulder and waist attachments, such as those discussed above, can be reduced or eliminated, relying solely on connecting strings, cords or narrow straps (e.g., see straps 642, 650) between the user and the top and bottom portions of the decoy pillow 630.

As shown, an exemplary decoy pillow 630 can comprise opposing top and bottom ends 632 and 634, wherein the top end 630 includes a mid-shoulder attachment means 631 for tethering to a user's upper body at a position between the shoulders. The mid-shoulder attachment means 631 can comprise a string or cord or any other type of connection having sufficient length to allow free rotation of the decoy pillow 630 between opposing positions along the user's right and left back sides in response to gravity. An opposing end of the mid-shoulder attachment means 631 can include a clip 633 attachable to a night garment at the user's neck or a string positioned at the user's neck for maintaining the referenced shoulder attachment position. Other similar attachment devices may be equally functional.

The bottom end 634 of the decoy pillow 630 can further comprise a mid-waist attachment means 635 for tethering to a user's low back at a centered position near the user's spine with sufficient length to allow free rotation of the decoy pillow 630 between the opposing positions along the user's right and left hack sides in response to gravity. Here again, this may be a clip, string or other attachment coupled at the bottom end 634 of the decoy pillow 630 at one end and at an opposing end to a waist garment or a belt 641 which can allow the decoy pillow 630 to fall to an appropriate simulated blocking position. Again, the function of the decoy pillow 630 is to generate the awareness of contact by the decoy pillow 630 at the user's back as a potential blocking or control position to trigger the conditioned response needed to cause the user to quickly arrest the progression of rolling to a supine position and to recover to a side sleeping position.

In a preferred embodiment, the mid-shoulder attachment means 631 and the mid-waist attachment means 635 can include adjustable, variable attachment lengths which can be sized to the body size of the user to more effectively stabilize the decoy pillow 630 in an approximate position along the user's spine while at the same time preserving free rotational displacement of the decoy pillow 630 to left and right back side, simulated blocking or control positions at proper displacement lengths to trigger the prior conditioned awareness. This conditioned awareness by the simulated blocking action of the decoy pillow 630 is sufficient to trigger a muscular response of the user to recover to the side sleeping position rather than progressing to a supine position during sleep.

In a further example, the decoy pillow 630 can be configured to comprise insufficient blocking contact with the user to actually prevent rotation to the supine position if the user choses to continue rotation over the decoy pillow. This may provide a lesser degree of restriction of rotational movement to the user compared to the highly restricted movement of the user with an actual blocking pillow used during training. This lesser restrictive condition creates a greater sense of freedom for improved comfort to the user while using the decoy pillow 630. Similarly, the dimensions of the decoy pillow 630 may be substantially smaller than the dimensions of the prior actual blocking pillow applied as part of the user's training, thereby substantially reducing a likelihood of discomfort or interference from body contact at an annoying or bothersome level arising from the presence of the decoy pillow 630 at the user's back during use because of its less obtrusive nature having a reduced comparative size.

Likewise, the weight of the decoy pillow 630 may be substantially less than the weight of the prior actual blocking pillow applied as part of the user's training, thereby also substantially reducing a likelihood of discomfort or interference from body contact with the user arising from the presence of the decoy pillow at the user's back during use because of its less obtrusive nature having a reduced comparative weight and size.

The effectiveness of the decoy pillow 630 can be further enhanced by use of a knee pillow 663 configured for positioning at knees of the user to bias the user in a side sleeping orientation when the knee pillow 663 is positioned between legs of the user. An attachment strap is useful to maintain proper positioning of the knee pillow 663 between the legs of the user, particularly during rotation from side to side.

Finally, further stability for a side sleeping orientation is facilitated with a chest pillow 665 configured for positioning at a front trunk position of the user to bias the user in a side sleeping orientation when the chest pillow 665 is held in position at a front trunk portion of the user. The combination of the knee pillow 663 and the chest pillow 665 tends to properly position the user in the preferred side orientation based on natural arm and leg positions which lie along a vertical orientation to the bed surface. In this state, as the user slightly shifts weight to commence rotation to a supine position, the decoy pillow 630 is quickly encountered, triggering the conditioned response and a rapid recovery to a side sleeping orientation.

With reference to FIG. 10, illustrated is a schematic diagram of an exemplary patient monitoring system (e.g., a remote patient monitoring system) operable with and incorporating any of the back-mounted support structures of FIGS. 1A-1B, 2-4, 6A-6B or 8A-8B, in accordance with one example of the present disclosure. The patient monitoring system 800 can comprise one or more back-mounted support devices, such as the back-mounted support device 810. The back-mounted support device 810 is intended to represent any type or configuration based on the technology disclosed herein. As such, the back-mounted support device 810 can comprise one or more shoulder attachment members 816, 818, one or more waist attachment members 816, 818, a stiffening member 850, optionally a shoulder and a waist platform 820, 830, a position control device (e.g., a. back pillow 830), and various monitoring devices 880 supported about any combination of these.

The patient monitoring system 800 can further be operable within a local network 902 or with a network connected mobile device 904, Indeed, each of the monitoring devices supported on the back-mounted support device 810 can comprise a transceiver that facilitates the receipt and transmission of data to/from the local network 902 (e.g., via a wireless router (not shown)) and/or to/from the mobile device 904. For example, data obtained by the one or more monitoring devices 880 can be transmitted to the local network 902 or to the mobile device 904 for purposes of storage within a database or further transmission.

The local network 902 and/or the mobile device 904 can be communicatively coupled to a broader communications network (e.g., a cellular network, a global Internet network, an intranet network, and any others). Data can be transmitted to the broader network for a variety of purposes. In one example, the patient monitoring system 800 can further comprise a data processing platform 908 communicatively coupled to the broader communications network 906, the data processing platform 908 being operable to receive the data obtained from the various monitoring devices 880 supported on the one or more back-mounted support devices 810 as transmitted thereto. Once the data is received at the data processing platform 908, the data can be processed, saved/stored and manipulated as needed or desired for one or more health related or research or other purposes. In one example, the data processing platform can comprise a remote patient monitoring platform.

With respect to the application of monitoring body parameters during sleep, the data processing platform can include a data processing component to record and save the data obtained during a measuring event. An additional comparator component can be included which is capable of identifying and recording subsequent patterns or recurrence of the predetermined positions of sleep during monitoring of an ongoing period of sleep of the user. The data processing platform may also include (i) a data processing component to record and save body positions of sleep of the user as part of a time referenced database and (ii) an additional component capable of grouping the saved body positions of various categories of side, supine and any intermediate rotated positions occurring during a period of sleep of the user during a given period of time, When used in connection with sleep apnea analysis, occurrence of apnea events can be synchronized in the record with exact body orientations to assist in identifying particular orientations that trigger breathing interruptions.

Additional sensors may be included providing access to additional data processing components which record and save predetermined body parameters such as blood pressure, pulse rates, oxygen saturation, abdominal respiration rates (for comparison with thoracic extension) and the like relating to sleep conditions unique to the user as part of a reference database. Other attendant functions may be added for recording and correlating the additional body parameters with respect to time indexed recurrence of the positions of sleep during monitoring of an ongoing period of sleep of the user. Appropriate blue tooth and remote accessing components may provide access for telemetry monitoring of sensor data.

Other methods, structural features and combinations thereof will become apparent to one of ordinary skill in the art, based on the foregoing examples. Accordingly, the present invention is to be construed only by the following claims, and is not to be limited to specific examples provided above.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Although the disclosure may not expressly disclose that some embodiments or features described herein may be combined with other embodiments or features described herein, this disclosure should be read to describe any such combinations that would be practicable by one of ordinary skill in the art. The use of “or” in this disclosure should be understood to mean non-exclusive or, i.e., “and/or,” unless otherwise indicated herein.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The following clauses are exemplary of the examples discussed herein:
1. A back-mounted support device for providing a stable platform for attachment of various monitoring and position control devices on a human body for enabling ongoing measurement of body parameters of an individual user located on a bed, as well as being adaptable for sustaining a desired body orientation on the bed, such as a side-sleeping position, said device comprising:
   an elongate stiffening member configured for placement along a user's spine, the stiffening member including a length and limited width and having (i) a top end to be positioned substantially between shoulder blades of the user and (ii) a bottom end to be positioned along the spine and near a waistline of the user, the limited width between the shoulder blades being configured to provide greater comfort to the user and maintenance of long term compliance in use, the stiffening member being further configured through use of modulus of elasticity and geometric shape for generally maintaining a stabilized position proximate at the user's back and along the spine;
   a shoulder attachment member configured for attachment at shoulders of the user and coupled to the top end of the stiffening member to stabilize an upper portion of the stiffening member in a position along the user's upper spine;
   a waist attachment member configured for attachment near the waist of the user and coupled to the bottom end of the stiffening member to stabilize a lower portion of the stiffening member in a position along the user's lower spine and in a substantially centered location of the user's low back, the shoulder attachment and waist attachment members being configured to stabilize the stiffening member substantially along the spine during sleep, while minimizing irritating body contact; and
   at least one body position and orientation sensor coupled to the stiffening member and the back-mounted support device in a substantially fixed positional relationship therewith and proximate to the user's back, the sensor being configured to monitor body movement and orientation with respect to various side-versus-supine positions of the body during sleep.
2. A device as defined in example 1, wherein the at least one body position sensor is mounted near the top end of the stiffening member and at the shoulder attachment member and or the waist attachment member.
3. A device as defined in example 1, further comprising at least one additional sensor mounted on the back mounted support device, said at least one additional sensor being selected from the group consisting of blood pressure, blood gas levels, oximetry, pulse rate, glucose levels. EKG, temperature, incontinence, respiration and a light source coupled to the position sand orientation sensor including means for activating the light source automatically when the position sensor registers that the user is standing or has fallen from bed.
4. A device as defined in example 1 for monitoring respiration rate of the user, the device including opposing tension sensor components, each having a tension line capable of measuring user abdominal movement associated with breathing with one end of each tension line being adapted for attachment along a medial anchor section of the stiffening member and an opposite end of the tension line to the opposing tension sensor components mounted at opposite belt portions of the harness and configured to monitor changing tension along the intermediate tension lines for registering abdominal movement in response to breath,
5. A device as defined in example 1, further including at least one airflow respiration monitoring sensor with a control component mounted at a location selected from at least one of the group consisting of (i) the waist attachment member GO the shoulder attachment member and (iii) the stiffening member with at least one remote sensor component being interconnected to the control component and respiration monitoring sensor by a tubular interconnect line mounted along the stiffening member and/or along the shoulder attachment member, wherein the remote sensor includes a nose canula for measuring air flow respiration of the user.
6. A device as defined in example 1, further comprising a remote tension sensor component including a tension sensor coupled to at least one thoracic or abdominal respiration band which is mounted at the stiffening member to provide a stable anchor point for the band with respect to the user and having an adjustable length which can be applied in tension around the user's chest or abdomen for monitoring respiration expansion.
7. A device as defined in example 1, wherein the back-mounted support device further includes upper and lower pillow attachment members at opposing ends of the stiffening member for attaching an elongate, tubular shaped pillow at opposing top and bottom ends thereof for connecting the tubular pillow at the user's back and along the spine, each of the upper and lower attachment members having a length which provides for free rotation and fall of the pillow between opposing positions along the user's back at right and left sides in response to gravity.
8. A back-mounted support device for enabling an individual user located on a bed to sustain a preferential side-sleeping orientation on either a left or right side, said device comprising:
   an elongate, tubular shaped pillow having opposing top and bottom ends and being attached to and supported by a light weight, elongate stiffening member configured for placement along a user's spine, the stiffening member including a length and limited width configured (i) to extend from a top end of the stiffening member to be positioned substantially between shoulder blades of the user (ii) to a bottom end to be positioned along the spine and near a waistline of the user, the limited width between the shoulder blades being configured to provide greater comfort to the user and maintenance of long term compliance in use, the stiffening member being further configured through use of modulus of elasticity and geometric shape for generally maintaining a stabilized position proximate at the user' back and along the spine;
   a top end of the elongate pillow being coupled at the top end of the stiffening member by a first attachment member having an attachment length which provides for free rotation of the pillow between opposing positions along the user's back at right and left sides in response to gravity;
   the bottom end of the elongate pillow being coupled at the bottom end of the stiffening member by a second attachment member having a coordinated attachment length with the first attachment member which provides for synchronized rotation of the pillow in response to gravity between the opposing back-contact positions near the user's right and left sides;
   a shoulder attachment member configured for attachment at shoulders of the user and coupled to the top end of the stiffening member to stabilize an upper portion of the stiffening member in a position along the user's spine; and
   a waist attachment member configured for attachment near the waist of the user and coupled to the bottom end of the stiffening member to stabilize a lower portion of the stiffening member in a position along the user's spine and in a substantially centered location of the user's low back, the shoulder attachment and waist attachment members being configured to stabilize the stiffening member substantially along the spine during sleep, while minimizing irritating body contact,
9. A device as defined in example 8, further comprising means for attaching a body position sensor to the stiffening member in a substantially fixed positional relationship and proximate to the user's back, the sensor being configured to monitor body movement and orientation with respect to various side-versus-supine positions of the body during sleep.
10. A device as defined in example 8, wherein the first and second attachment members coupled at the top and bottom ends of the pillow include a flexible, variable attachment length which provides for selective free rotation of the pillow between various opposing positions along the user's right and left sides in response to gravity.
11. A device as defined in example 8, wherein the stiffening member includes a foam insert within an enclosing sleeve which resists substantial twisting away from the user's back during use and is configured at the top end to nest at the individual's back substantially between the shoulder blades.
12. A device as defined in example 8, wherein the shoulder attachment member comprises a pair of shoulder straps connected at one end to the top end of the stiffening member for positioning the straps to extend over shoulders of the user and attach at opposing strap ends at the waist attachment member near opposing sides of and near the bottom end of the stiffening member, creating a harness configuration to support the device in a stable position substantially centered on the user's back.
13. A device as defined in example 8, further comprising at least two spacer elements positioned at separated locations on the stiffening member and oriented for contact at the user's back to thereby space the stiffening member free of direct contact along a substantial length of the stiffening member to reduce potential discomfort from contact of the stiffening members with the user's back.
14. A device as defined in example 8, further comprising an orientation sensor attached to the stiffening members in general planer, parallel relationship with the stiffening member to provide reference data regarding body orientation of the user indicative of a sleep posture in a respective left side, supine, right side and/or frontal body orientation on a bed surface.
15. A device as defined in example 14, wherein the orientation sensor is coupled to (i) a data processing component to record and save predetermined positions of sleep of the user as part of a reference data base, and (ii) an additional comparator component capable of identifying and recording subsequent recurrence of the predetermined positions of sleep during monitoring of an ongoing period of sleep of the user.
16. A device as defined in example 14, wherein the orientation sensor includes (i) a data processing component to record and save body positions of sleep of the user as part of a time referenced data base and (ii) an additional component capable of grouping the saved body positions of various categories of side, supine and any intermediate rotated positions occurring during a period of sleep of the user during a given period of time.
17. A device as defined in example 14, further comprising at least one additional sensor selected from the group consisting of blood pressure, blood gas levels, oximetry, pulse rate, glucose levels. EKG, temperature, incontinence, respiration wherein the at least one additional sensor is coupled to additional data processing components which record and save additional predetermined body parameters selected. from the group consisting of unique to the user as part of a reference data base, including access to an additional comparator component capable of identifying, recording and correlating the additional body parameters with respect to time indexed recurrence of the positions of sleep detected during monitoring of an ongoing period of sleep of the user.
18. A device as defined in example 14, wherein the stiffening member comprises a light weight, stiffening rod which resists rotational twisting along its longitudinal axis and away from the user's back during use and is configured at the top end to substantially nest at the individual's back between the shoulder blades.
19. A device as defined in example 18, wherein the stiffening rod comprises a fiber composite composition having an upper flexible end section which can be biased into a slight curvature within a shorter section of the sleeve to correspond to curvature of an upper back portion of the user and a. bottom end of greater stiffness to provide a stable anchor position at the waist attachment member to maintain a substantially centered alignment with the spine.
20. A device as defined in example 18, wherein the shoulder attachment member comprises a pair of shoulder straps connected at one end to the top end of the stiffening rod and positioned to extend over shoulders of the user and attach at opposing strap ends at the waist attachment member near opposing sides of and near the bottom end of the stiffening rod, creating a harness configuration to support the device in a stable position centered on the user's back.
21. A device as defined in example 18, further comprising at least two spacer elements positioned at separated locations on the stiffening rod and oriented for contact at the user's back to thereby space the stiffening rod out of direct contact with the user's back along a substantial length of the stiffening rod to reduce potential discomfort from contact of the stiffening rod with the user's back.
22. A device as defined in example 18, wherein the stiffening rod further comprises:
   at least two short transverse axial stabilizing members rigidly coupled to and positioned towards opposing ends of the stiffening rod in co-planar relationship, substantially parallel with the user's back; and
   at least one transverse stabilizing member attached to the stiffening rod between the axial stabilizing members and along a common plane therewith to define a sensor mounting base substantially parallel with the user's back and configured to resist rotational twisting along a longitudinal axis of the rod and away from the user's back during sleep.
23. A device as defined in example 18, further comprising an orientation sensor attached in planar relationship to the sensor mounting base to provide reference data regarding body orientation of the user indicative of a sleep posture in respective left side, supine, right side and/or frontal body orientation on a bed surface.
24. A device as defined in example 18, wherein the first attachment member includes a variable attachment length which provides for selective free rotation of the pillow between various opposing positions along the user's right and left sides in response to gravity, the bottom end of the elongate pillow being coupled at the bottom end of the stiffening rod by a second attachment member having a variable attachment length Which provides for the selective free rotation of the pillow between the various opposing positions along the user's right and left sides in response to gravity.
25. A back-mountable, rotatable pillow device for enabling an individual located on a bed to sustain a preferential side-sleeping orientation on either a left or right side as opposed to a supine body orientation, said device comprising:
   an elongate stiffening member being configured for stable positioning along the user's spine and having (i) a top end to be positioned substantially between the user's shoulder blades and (ii) a bottom end to be positioned near a waistline of the user, said stiffening member having a material composition and geometrical shape capable of maintaining the stable positioning to avoid excessive twisting and buckling during normal body movement during sleep;
   an elongate, tubular shaped pillow having opposing top and bottom ends and being attached to and supported by the stiffening member and configured for placement along the user's spine, the top end of the elongate pillow being coupled to the top end of the stiffening member by a first interconnecting attachment member having an attachment length which provides for free rotation of the pillow between opposing positions along the user's right and left back sides in response to gravity, the bottom end of the elongate pillow being coupled at the bottom end of the stiffening member by a second interconnecting attachment member having an attachment length which provides for the rotation of the pillow between the opposing positions along the user's right and left back sides;
   a shoulder attachment member configured for attachment at shoulders of the user and coupled to the top end of the stiffening member and configured to stabilize an upper portion of the stiffening member in a position along the user's spine; and
   a waist attachment member configured for attachment near the waist of the user and coupled to the bottom end of the stiffening member and configured to stabilize a lower portion of the stiffening member in a position along the user's spine in an approximate centered location of the user's low back;
   the shoulder attachment and waist attachment members being cooperatively configured to stabilize the position of the stiffening member along the spine during sleep, while at the same time preserving free rotational displacement of the pillow to left and right back side blocking positions at proper displacement lengths to minimize the user's body rotation to a supine sleeping orientation.
26. A device as defined in example 25, wherein the first attachment member includes a variable attachment length which provides for selective free rotation of the pillow between various opposing positions along the user's right and left sides in response to gravity, the bottom end of the elongate pillow being coupled at the bottom end of the stiffening member by a second attachment member having a variable attachment length which provides for the selective free rotation of the pillow between the various opposing positions along the user's right and left back sides in response to gravity,
27. A back-mountable, rotatable decoy pillow device for enabling an individual who has been trained to favor side sleeping by repeated use of prior actual blocking action of a captured, rotatable pillow between (i) a bed surface against one side of the pillow and (ii) an individual's left or right back side at an opposing side of the pillow, said training having provided the individual with a conditioned mental awareness of this prior actual blocking action for impeding rotation of the user's body to a supine position during sheep, said decoy pillow device comprising:
   an elongate, tubular shaped pillow having opposing top and bottom ends, (i) the top end having mid-shoulder attachment means including an adjustable length for tethering to a user's upper body at a position between the shoulders with sufficient length to allow free rotation of the pillow between opposing positions along the user's right and left back sides in response to gravity, (ii) the bottom end having mid-waist adjustable attachment means for tethering to a user's low back at a substantially centered position near the user's spine with sufficient length to allow free rotation of the decoy pillow between the opposing positions along the user's right and left back sides in response to gravity to generate an awareness of contact of the decoy pillow at the individual's back as a potential blocking position to potentially block rotation of the user from a side sleeping position to a supine position.
28. A device as defined in example 27, the mid-shoulder attachment means being configured to stabilize an upper portion thereof in an approximate position along the user's upper spine toward the users neck and the mid-waist attachment means being configured to stabilize a lower portion thereof in an approximate position along the user's lower spine in a substantially centered location of the user's low back while at the same time preserving free rotational displacement of the pillow to left and right back side simulated blocking positions at proper displacement lengths to trigger the prior conditioned mental awareness previously generated with the actual prior blocking action of a captured, rotatable pillow between (i) a bed surface against one side of the pillow and (ii) an individual's left or right back side at an opposing side of the pillow, said simulated conditioned awareness of the blocking action of the decoy pillow being sufficient to immediately trigger muscular response of the user to recover to the side sleeping position rather than progressing to a supine position during sleep.
29. A device as defined in example 27, wherein blocking action of the decoy pillow provides insufficient blocking resistance to actually prevent rotation to the supine position if the user choses to continue rotation over the decoy pillow, thereby allowing the user to ignore the decoy pillow and enjoy a greater sense of freedom of movement and improved comfort compared to an actual blocking pillow.
30. A device as defined in example 27, wherein radial dimensions of the decoy pillow are substantially smaller than radial dimensions of the prior actual blocking pillow applied as part of the training which contributed to development of the conditioned awareness present as a result of the repeated use of actual blocking action of the actual captured, rotatable pillow, thereby substantially reducing discomfort or interference from body contact at an annoying level with the user arising from presence of the decoy pillow at the user's back during use because of its less obtrusive nature having a reduced comparative size.
31. A device as defined in example 27, wherein weight of the decoy pillow is substantially less than weight of the prior actual blocking pillow applied as part of the training which contributed to development of the conditioned awareness present as a result of the repeated use of actual blocking action of the actual captured, rotatable pillow, thereby substantially reducing discomfort or interference from body contact with the user arising from presence of the decoy pillow at the user's back during use because of its less obtrusive nature having a reduced comparative weight.
32. A device as defined in example 27, wherein mid-shoulder attachment means includes a variable attachment length which provides for selective free rotation of the pillow between various opposing positions along the user's right and left sides in response to gravity, the mid-waist attachment means having a variable attachment length which provides for the selective free rotation of the pillow between the various opposing positions along the user's right and left back sides in response to gravity.
33. A device as defined in example 27, further comprising a knee pillow configured for positioning at knees of the user to bias the user in a side sleeping orientation when the pillow is positioned between legs of the user and to complement the conditioned awareness which favors a user choice of side sleeping orientation over a supine position.
34. A device as defined in example 27, further comprising a chest pillow configured for positioning at a front trunk position of the user to bias the user in a side sleeping orientation when the pillow is held in position at a front trunk portion of the user and to complement the conditioned awareness which favors a user choice of side sleeping orientation over a supine position.

What is claimed is:

1. A back-mounted support device for providing a stable platform for attachment of various monitoring and position control devices on a human body for enabling ongoing measurement of body parameters of an individual user located on a bed, as well as being adaptable for sustaining a desired body orientation on the bed, such as a side-sleeping position, said device comprising:
an elongate stiffening member configured for placement along a user's spine, the stiffening member including a length and limited width and having (i) a top end to be positioned substantially between shoulder blades of the user and (ii) a bottom end to be positioned along the spine and near a waistline of the user, the limited width between the shoulder blades being configured to provide greater comfort to the user and maintenance of long term compliance in use, the stiffening member being further configured through use of modulus of elasticity and geometric shape for generally maintaining a stabilized position proximate at the user's back and along the spine;
a shoulder attachment member configured for attachment at shoulders of the user and coupled to the top end of the stiffening member to stabilize an upper portion of the stiffening member in a position along the user's upper spine;
a waist attachment member configured for attachment near the waist of the user and coupled to the bottom end of the stiffening member to stabilize a lower portion of the stiffening member in a position along the user's lower spine and in a substantially centered location of the user's low back, the shoulder attachment and waist attachment members being configured to stabilize the stiffening member substantially along the spine during sleep, while minimizing irritating body contact; and
at least one body position and orientation sensor coupled to the stiffening member and the back-mounted support device in a substantially fixed positional relationship therewith and proximate to the user's back, the sensor being configured to monitor body movement and orientation with respect to various side-versus-supine positions of the body during sleep.

2. A device as defined in claim 1, wherein the at least one body position sensor is mounted near the top end of the stiffening member and at the shoulder attachment member and or the waist attachment member.

3. A device as defined in claim 1, further comprising at least one additional sensor mounted on the back mounted support device, said at least one additional sensor being selected from the group consisting of blood pressure, blood gas levels, oximetry, pulse rate, glucose levels, EKG-, temperature, incontinence, respiration and a light source coupled to the position sand orientation sensor including means for activating the light source automatically when the position sensor registers that the user is standing or has fallen from bed.

4. A device as defined in claim 1 for monitoring respiration rate of the user, the device including opposing tension sensor components, each having a tension line capable of measuring user abdominal movement associated with breathing with one end of each tension line being adapted for attachment along a medial anchor section of the stiffening member and an opposite end of the tension line to the opposing tension sensor components mounted at opposite belt portions of the harness and configured to monitor changing tension along the intermediate tension lines for registering abdominal movement in response to breath.

5. A device as defined in claim 1, further including at least one airflow respiration monitoring sensor with a control component mounted at a location selected from at least one of the group consisting of (i) the waist attachment member (ii) the shoulder attachment member and (iii) the stiffening member with at least one remote sensor component being interconnected to the control component and respiration monitoring sensor by a tubular interconnect line mounted along the stiffening member and/or along the shoulder attachment member, wherein the remote sensor includes a nose canula for measuring air flow respiration of the user.

6. A device as defined in claim 1, further comprising a remote tension sensor component including a tension sensor coupled to at least one thoracic or abdominal respiration band which is mounted at the stiffening member to provide a stable anchor point for the band with respect to the user and having an adjustable length which can be applied in tension around the user's chest or abdomen for monitoring respiration expansion.

7. A device as defined in claim 1, wherein the back-mounted support device further includes upper and lower pillow attachment members at opposing ends of the stiffening member for attaching an elongate, tubular shaped pillow at opposing top and bottom ends thereof for connecting the tubular pillow at the user's back and along the spine, each of the upper and lower attachment members having a length which provides for free rotation and fall of the pillow between opposing positions along the user's back at right and left sides in response to gravity.

8. A back-mounted support device for enabling an individual user located on a bed to sustain a preferential side-sleeping orientation on either a left or right side, said device comprising:
an elongate, tubular shaped pillow having opposing top and bottom ends and being attached to and supported by a light weight, elongate stiffening member configured for placement along a user's spine, the stiffening member including a length and limited width configured (i) to extend from a top end of the stiffening member to be positioned substantially between shoulder blades of the user i.ii i to a bottom end to be positioned along the spine and near a waistline of the user, the limited width between the shoulder blades being configured to provide greater comfort to the user and maintenance of long term compliance in use, the stiffening member being further configured through use of modulus of elasticity and geometric shape for generally maintaining a stabilized position proximate at the user' back and along the spine;
a top end of the elongate pillow being coupled at the top end of the stiffening member by a first attachment member having an attachment length which provides for free rotation of the pillow between opposing positions along the user's back at right and left sides in response to gravity;
the bottom end of the elongate pillow being coupled at the bottom end of the stiffening member by a second attachment member having a coordinated attachment length with the first attachment member which provides for synchronized rotation of the pillow in response to gravity between the opposing back-contact positions near the user's right and left sides;
a shoulder attachment member configured for attachment at shoulders of the user and coupled to the top end of the stiffening member to stabilize an upper portion of the stiffening member in a position along the user's spine; and
a waist attachment member configured for attachment near the waist of the user and coupled to the bottom end of the stiffening member to stabilize a lower portion of the stiffening member in a position along the user's spine and in a substantially centered location of the user's low back, the shoulder attachment and waist attachment members being configured to stabilize the stiffening member substantially along the spine during sleep, while minimizing irritating body contact.

9. A device as defined in claim 8, further comprising means for attaching a body position sensor to the stiffening member in a substantially fixed positional relationship and proximate to the user's back, the sensor being configured to monitor body movement and orientation with respect to various side-versus-supine positions of the body during sleep.

10. A device as defined in claim 8, wherein the first and second attachment members coupled at the top and bottom ends of the pillow include a flexible, variable attachment length which provides for selective free rotation of the pillow between various opposing positions along the user's right and left sides in response to gravity.

11. A device as defined in claim 8, wherein the stiffening member includes a foam insert within an enclosing sleeve which resists substantial twisting away from the user's back during use and is configured at the top end to nest at the individual's back substantially between the shoulder blades.

12. A device as defined in claim 8, wherein the shoulder attachment member comprises a pair of shoulder straps connected at one end to the top end of the stiffening member for positioning the straps to extend over shoulders of the user and attach at opposing strap ends at the waist attachment member near opposing sides of and near the bottom end of the stiffening member, creating a harness configuration to support the device in a stable position substantially centered on the user's back.

13. A device as defined in claim 8, further comprising at least two spacer elements positioned at separated locations on the stiffening member and oriented for contact at the user's back to thereby space the stiffening member free of direct contact along a substantial length of the stiffening member to reduce potential discomfort from contact of the stiffening members with the user's back.

14. A device as defined in claim 8, further comprising an orientation sensor attached to the stiffening members in general planer, parallel relationship with the stiffening member to provide reference data regarding body orientation of the user indicative of a sleep posture in a respective left side, supine, right side and/or frontal body orientation on a bed surface.

15. A device as defined in claim 14, wherein the orientation sensor is coupled to (i) a data processing component to record and save predetermined positions of sleep of the user as part of a reference data base, and (ii) an additional comparator component capable of identifying and recording subsequent recurrence of the predetermined positions of sleep during monitoring of an ongoing period of sleep of the user.

16. A device as defined in claim 14, wherein the orientation sensor includes (i) a data processing component to record and save body positions of sleep of the user as part of a time referenced data base and GO an additional component capable of grouping the saved body positions of various categories of side, supine and any intermediate rotated positions occurring during a period of sleep of the user during a given period of time.

17. A device as defined in claim 14, further comprising at least one additional sensor selected from the group consisting of blood pressure, blood gas levels, oximetry, pulse rate, glucose levels, EKG, temperature, incontinence, respiration wherein the at least one additional sensor is coupled to additional data. processing components which record and save additional predetermined body parameters selected from the group consisting of unique to the user as part of a reference data base, including access to an additional comparator component capable of identifying, recording and correlating the additional body parameters with respect to time indexed recurrence of the positions of sleep detected during monitoring of an ongoing period of sleep of the user.

18. A device as defined in claim 14, wherein the stiffening member comprises a light weight, stiffening rod which resists rotational twisting along its longitudinal axis and away from the user's back during use and is configured at the top end to substantially nest at the individual's back between the shoulder blades.

19. A device as defined in claim 18, wherein the stiffening rod comprises a fiber composite composition having an upper flexible end section which can be biased into a slight curvature within a shorter section of the sleeve to correspond to curvature of an upper back portion of the user and a bottom end of greater stiffness to provide a stable anchor position at the waist attachment member to maintain a substantially centered alignment with the spine.

70. A device as defined in claim 18, wherein the shoulder attachment member comprises a pair of shoulder straps connected at one end to the top end of the stiffening rod and positioned to extend over shoulders of the user and attach at opposing strap ends at the waist attachment member near opposing sides of and near the bottom end of the stiffening rod, creating a harness configuration to support the device in a stable position centered on the user's back.

21. A device as defined in claim 18, further comprising at least two spacer elements positioned at separated locations on the stiffening rod and oriented for contact at the user's back to thereby space the stiffening rod out of direct contact with the user's back along a substantial length of the stiffening rod to reduce potential discomfort from contact of the stiffening rod with the user's back.

22. A device as defined in claim 18, wherein the stiffening rod further comprises:
at least two short transverse axial stabilizing members rigidly coupled to and positioned towards opposing ends of the stiffening rod in co-planar relationship, substantially parallel with the user's back; and
at least one transverse stabilizing member attached to the stiffening rod between the axial stabilizing members and along a common plane therewith to define a sensor mounting base substantially parallel with the user's back and configured to resist rotational twisting along a longitudinal axis of the rod and away from the user's back during sleep.

23. A device as defined in claim 18, further comprising an orientation sensor attached in planar relationship to the sensor mounting base to provide reference data regarding body orientation of the user indicative of a sleep posture in respective left side, supine, right side and/or frontal body orientation on a bed surface.

24. A device as defined in claim 18, wherein the first attachment member includes a. variable attachment length which provides for selective free rotation of the pillow between various opposing positions along the user's right and left sides in response to gravity, the bottom end of the elongate pillow being coupled at the bottom end of the stiffening rod by a second attachment member having a variable attachment length which provides for the selective free rotation of the pillow between the various opposing positions along the user's right and left sides in response to gravity.

25. A back-mountable, rotatable pillow device for enabling an individual located on a bed to sustain a preferential side-sleeping orientation on either a left or right side as opposed to a supine body orientation, said device comprising:
an elongate stiffening member being configured for stable positioning along the user's spine and having (i) a top end to be positioned substantially between the user's shoulder blades and (ii) a bottom end to be positioned near a waistline of the user, said stiffening member having a material composition and geometrical shape capable of maintaining the stable positioning to avoid excessive twisting and buckling during normal body movement during sleep;
an elongate, tubular shaped pillow having opposing top and bottom ends and being attached to and supported by the stiffening member and configured for placement along the user's spine, the top end of the elongate pillow being coupled to the top end of the stiffening member by a first interconnecting attachment member having an attachment length which provides for free rotation of the pillow between opposing positions along the user's right and left back sides in response to gravity, the bottom end of the elongate pillow being coupled at the bottom end of the stiffening member by a second interconnecting attachment member having an attachment length which provides for the rotation of the pillow between the opposing positions along the user's right and left back sides;
a shoulder attachment member configured for attachment at shoulders of the user and coupled to the top end of the stiffening member and configured to stabilize an upper portion of the stiffening member in a position along the user's spine; and
a waist attachment member configured for attachment near the waist of the user and coupled to the bottom end of the stiffening member and configured to stabilize a lower portion of the stiffening member in a position along the user's spine in an approximate centered location of the user's low back;
the shoulder attachment and waist attachment members being cooperatively configured to stabilize the position of the stiffening member along the spine during sleep, while at the same time preserving free rotational displacement of the pillow to left and right back side blocking positions at proper displacement lengths to minimize the user's body rotation to a supine sleeping orientation.

26. A device as defined in claim 25, wherein the first attachment member includes a variable attachment length which provides for selective free rotation of the pillow between various opposing positions along the user's right and left sides in response to gravity, the bottom end of the elongate pillow being coupled at the bottom end of the stiffening member by a second attachment member having a variable attachment length which provides for the selective free rotation of the pillow between the various opposing positions along the user's right and left back sides in response to gravity.

27. A back-mountable, rotatable decoy pillow device for enabling an individual who has been trained to favor side sleeping by repeated use of prior actual blocking action of a captured, rotatable pillow between (i) a bed surface against one side of the pillow and an individuals left or right back side at an opposing side of the pillow, said training having provided the individual with a conditioned mental awareness of this prior actual blocking action for impeding rotation of the user's body to a supine position during sleep, said decoy pillow device comprising:
an elongate, tubular shaped pillow having opposing top and bottom ends, (i) the top end having mid-shoulder attachment means including an adjustable length for tethering to a user's upper body at a position between the shoulders with sufficient length to allow free rotation of the pillow between opposing positions along the user's right and left back sides in response to gravity, (ii) the bottom end having mid-waist adjustable attachment means for tethering to a user's low back at a substantially centered position near the user's spine with sufficient length to allow free rotation of the decoy pillow between the opposing positions along the user's right and left back sides in response to gravity to generate an awareness of contact of the decoy pillow at the individual's back as a potential blocking position to potentially block rotation of the user from a side sleeping position to a supine position.

28. A device as defined in claim 27, the mid-shoulder attachment means being configured to stabilize an upper portion thereof in an approximate position along the user's upper spine toward the users neck and the mid-waist attachment means being configured to stabilize a lower portion thereof in an approximate position along the user's lower spine in a substantially centered location of the user's low back while at the same time preserving free rotational displacement of the pillow to left and right back side simulated blocking positions at proper displacement lengths to trigger the prior conditioned mental awareness previously generated with the actual prior blocking action of a captured, rotatable pillow between (i) a bed surface against one side of the pillow and (ii) an individual's left or right back side at an opposing side of the pillow, said simulated conditioned awareness of the blocking action of the decoy pillow being sufficient to immediately trigger muscular response of the user to recover to the side sleeping position rather than progressing to a supine position during sleep.

29. A device as defined in claim 27, wherein blocking action of the decoy pillow provides insufficient blocking resistance to actually prevent rotation to the supine position if the user choses to continue rotation over the decoy pillow, thereby allowing the user to ignore the decoy pillow and enjoy a greater sense of freedom of movement and improved comfort compared to an actual blocking pillow.

30. A device as defined in claim 27, wherein radial dimensions of the decoy pillow are substantially smaller than radial dimensions of the prior actual blocking pillow applied as part of the training which contributed to development of the conditioned awareness present as a result of the repeated use of actual blocking action of the actual captured, rotatable pillow, thereby substantially reducing discomfort or interference from body contact at an annoying level with the user arising from presence of the decoy pillow at the user's back during use because of its less obtrusive nature having a reduced comparative size.

31. A device as defined in claim 27, wherein weight of the decoy pillow is substantially less than weight of the prior actual blocking pillow applied as part of the training which contributed to development of the conditioned awareness present as a result of the repeated use of actual blocking action of the actual captured, rotatable pillow, thereby substantially reducing discomfort or interference from body contact with the user arising from presence of the decoy pillow at the user's back during use because of its less obtrusive nature having a reduced comparative weight.

32. A device as defined in claim 27, wherein mid-shoulder attachment means includes a variable attachment length which provides for selective free rotation of the pillow between various opposing positions along the user's right and left sides in response to gravity, the mid-waist attachment means having a variable attachment length which provides for the selective free rotation of the pillow between the various opposing positions along the user's right and left back sides in response to gravity.

33. A device as defined in claim 27, further comprising a knee pillow configured for positioning at knees of the user to bias the user in a side sleeping orientation when the pillow is positioned between legs of the user and to complement the conditioned awareness which favors a user choice of side sleeping orientation over a supine position.

34. A device as defined in claim 27, further comprising a chest pillow configured for positioning at a front trunk position of the user to bias the user in a side sleeping orientation when the pillow is held in position at a front trunk portion of the user and to complement the conditioned awareness which favors a user choice of side sleeping orientation over a supine position.
